(19) United States
(12) Patent Application Publication
(10) Pub. No.: US 2017/0324114 A1
(43) Pub. Date: Nov. 9, 2017

(54) ELECTROLYTIC SOLUTION

(71) Applicant: THE UNIVERSITY OF TOKYO, Tokyo (JP)

(72) Inventors: Tomoyuki KAWAI, Kariya-shi (JP); Junichi NIWA, Kariya-shi (JP); Atsuo YAMADA, Tokyo (JP); Yuki YAMADA, Tokyo (JP)

(73) Assignee: THE UNIVERSITY OF TOKYO, Tokyo (JP)

(21) Appl. No.: 15/527,575

(22) PCT Filed: Oct. 5, 2015
(86) PCT No.: PCT/JP2015/005052

§ 371 Date: May 17, 2017

(30) Foreign Application Priority Data
Nov. 18, 2014 (JP) 2014-233460
Mar. 10, 2015 (JP) 2015-047052

(51) Int. Cl. H01M 10/0564; H01M 10/0525; C07C 69/96

(52) U.S. Cl. CPC H01M 10/0564; H01M 10/0525; C07C 69/96; H01M 2300/0034

(57) ABSTRACT

An electrolytic solution including
a heteroelement-containing organic solvent at a mole ratio of not greater than 1.5 relative to a metal salt,
the heteroelement-containing organic solvent containing a linear carbonate represented by general formula (1) below,
the metal salt being a metal salt whose cation is an alkali metal, an alkaline earth metal, or aluminum and whose anion has a chemical structure represented by general formula (2) below.

R¹⁰OCOOR¹¹ general formula (1)

(R²¹X²¹)(R²²SO₂)N general formula (2)

### TECHNICAL FIELD

The present invention relates to an electrolytic solution to be used in power storage devices such as secondary batteries.

### BACKGROUND ART

Generally, a power storage device such as a secondary battery includes, as main components, a positive electrode, a negative electrode, and an electrolytic solution. In the electrolytic solution, an appropriate electrolyte is added at an appropriate concentration range. For example, in an electrolytic solution of a lithium ion secondary battery, a lithium salt such as LiClO₄, LiAsF₆, LiPF₆, LiBF₄, CF₃SO₃Li, and (CF₃SO₂)₂NLi is commonly added as an electrolyte, and the concentration of the lithium salt in the electrolytic solution is generally set at about 1 mol/L.

In an organic solvent to be used in an electrolytic solution, an organic solvent having a high dipole moment and a high relative permittivity such as ethylene carbonate or propylene carbonate is generally mixed by not less than about 30 vol %, in order to suitably dissolve an electrolyte.

Actually, Patent Literature 1 discloses a lithium ion secondary battery using an electrolytic solution that uses a mixed organic solvent containing ethylene carbonate by 33 vol % and that contains LiPF₆ at a concentration of 1 mol/L. Furthermore, Patent Literature 2 discloses a lithium ion secondary battery using an electrolytic solution that uses a mixed organic solvent containing ethylene carbonate and propylene carbonate by 66 vol % and that contains (CF₃SO₂)₂NLi at a concentration of 1 mol/L.

In addition, for the purpose of improving performance of secondary batteries, studies are actively conducted for various additives to be added to an electrolytic solution containing a lithium salt.

For example, Patent Literature 3 describes an electrolytic solution obtained by adding a small amount of a specific additive to an electrolytic solution that uses a mixed organic solvent containing ethylene carbonate by 30 vol % and that contains LiPF₆ at a concentration of 1 mol/L. Patent Literature 3 discloses a lithium ion secondary battery using this electrolytic solution. Furthermore, Patent Literature 4 describes an electrolytic solution obtained by adding a small amount of phenyl glycidyl ether to a solution that uses a mixed organic solvent containing ethylene carbonate by 30 vol % and that contains LiPF₆ at a concentration of 1 mol/L. Patent Literature 4 discloses a lithium ion secondary battery using this electrolytic solution.

### CITATION LIST

### Patent Literature

Patent Literature 1: JP2013149477(A)

Patent Literature 2: JP2013134922(A)

Patent Literature 3: JP2013145724(A)

Patent Literature 4: JP2013137873(A)

### SUMMARY OF INVENTION

### Technical Problem

As described in Patent Literature 1 to 4, conventionally, with respect to an electrolytic solution used in a lithium ion secondary battery, using a mixed organic solvent that contains, by not less than about 30 vol %, an organic solvent having a high relative permittivity and a high dipole moment such as ethylene carbonate or propylene carbonate and containing a lithium salt at a concentration of about 1 mol/L were technical common knowledge. In addition, as described in Patent Literature 3 to 4, studies for improving electrolytic solutions have been generally conducted with a focus on additives, which are separate from the lithium salt.

Unlike the focus of a person skilled in the art hitherto, the present invention relates to an electrolytic solution focused on: combining a metal salt which is a specific electrolyte and a linear carbonate having a low relative permittivity and a low dipole moment; and the mole ratio thereof. A purpose of the present invention is to newly provide a suitable electrolytic solution.

### Solution to Problem

The present inventors have conducted thorough investigation with much trial and error, without being confined to conventional technical common knowledge. As a result, the present inventors have found that a metal salt which is a specific electrolyte is dissolved at a significantly high concentration in a linear carbonate having a low relative permittivity and a low dipole moment. Furthermore, the present inventors have found that an electrolytic solution in which the mole ratio of a linear carbonate relative to a metal salt which is a specific electrolyte is in a specific range is suitably used in a power storage device such as a secondary battery. On the basis of these findings, the present inventors have completed the present invention.

An electrolytic solution of the present invention contains

a heteroelement-containing organic solvent at a mole ratio of not greater than 1.5 relative to a metal salt,

the heteroelement-containing organic solvent containing a linear carbonate represented by general formula (1) below,

the metal salt being a metal salt whose cation is an alkali metal, an alkaline earth metal, or aluminum and whose anion has a chemical structure represented by general formula (2) below.

R¹⁰OCOOR¹¹ general formula (1)

(R¹⁰ and R¹¹ are each independently selected from CₙHₐF_{b}Cl_{c}Br_{d}Iₑ that is a linear alkyl, or CₘH_{f}F_{g}ClₕBrᵢIⱼ that includes a cyclic alkyl in a chemical structure thereof. “n” is an integer not smaller than 1, “m” is an integer not smaller than 3, and “a”, “b”, “c”, “d”, “e”, “f”, “g”, “h”, “i”, and “j” are each independently an integer not smaller than 0 and satisfy 2n+1=a+b+c+d+e and 2m=f+g+h+i+j.)

(R²¹X²¹)(R²²SO₂)N general formula (2)

(R²¹ is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.

R²² is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.

R²¹ and R²² optionally bind with each other to form a ring.

X²¹ is selected from SO₂, C═O, C═S, R^{a}P═O, R^{b}P═S, S═O, or Si═O.

R^{a} and R^{b} are each independently selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; OH; SH; CN; SCN; or OCN.

R^{a} and R^{b} each optionally bind with R²¹ or R²² to form a ring.)

### Advantageous Effects of Invention

The new electrolytic solution of the present invention is suitable as an electrolytic solution for power storage devices such as secondary batteries.

### BRIEF DESCRIPTION OF DRAWINGS

FIG. 1 is a Raman spectrum chart obtained in Evaluation Example 4;

FIG. 2 is a graph of potential and response current obtained through linear sweep voltammetry performed in Evaluation Example A;

FIG. 3 shows a graph of FIG. 2 in an enlarged manner;

FIG. 4 is a graph showing the capacity retention rate of each lithium ion secondary battery in Evaluation Example I;

FIG. 5 shows charging and discharging curves of the lithium ion secondary battery of Example II in Evaluation Example I; and

FIG. 6 shows charging and discharging curves of the lithium ion secondary battery of Comparative Example I in Evaluation Example I.

### DESCRIPTION OF EMBODIMENTS

The following describes embodiments of the present invention. Unless mentioned otherwise in particular, a numerical value range of “a to b” described in the present specification includes, in the range thereof, a lower limit “a” and an upper limit “b”. A numerical value range is formed by arbitrarily combining such upper limit values, lower limit values, and numerical values described in Examples. In addition, numerical values arbitrarily selected within a numerical value range may be used as upper limit and lower limit numerical values.

An electrolytic solution of the present invention contains a heteroelement-containing organic solvent at a mole ratio of not greater than 1.5 relative to a metal salt,

the heteroelement-containing organic solvent containing a linear carbonate represented by general formula (1) below,

the metal salt being a metal salt whose cation is an alkali metal, an alkaline earth metal, or aluminum and whose anion has a chemical structure represented by general formula (2) below.

R¹⁰OCOOR¹¹ general formula (1)

(R¹⁰ and R¹¹ are each independently selected from CₙHₐF_{b}Cl_{c}Br_{d}Iₑ that is a linear alkyl, or CₘH_{f}F_{g}ClₕBrᵢIⱼ that includes a cyclic alkyl in a chemical structure thereof. “n” is an integer not smaller than 1, “m” is an integer not smaller than 3, and “a”, “b”, “c”, “d”, “e”, “f”, “g”, “h”, “i”, and “j” are each independently an integer not smaller than 0 and satisfy 2n+1=a+b+c+d+e and 2m=f+g+h+i+j.)

(R²¹X²¹)(R²²SO₂)N general formula (2)

(R²¹ is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.

R²² is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.

R²¹ and R²² optionally bind with each other to form a ring.

X²¹ is selected from SO₂, C═O, C═S, R^{a}P═O, R^{b}P═S, S═O, or Si═O.

R^{a} and R^{b} are each independently selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; OH; SH; CN; SCN; or OCN.

R^{a} and R^{b} each optionally bind with R²¹ or R²² to form a ring.)

As another mode of the electrolytic solution of the present invention, an electrolytic solution is understood that contains a linear carbonate at a mole ratio of not greater than 1.5 relative to a metal salt, the linear carbonate being represented by general formula (1) above, the metal salt being a metal salt whose cation is an alkali metal, an alkaline earth metal, or aluminum and whose anion has a chemical structure represented by general formula (2) above.

Among the linear carbonates represented by general formula (1) above, those represented by general formula (1-1) below are preferable.

R³OCOOR⁴ general formula (1-1)

(R¹³ and R¹⁴ are each independently selected from CₙHₐF_{b} that is a linear alkyl or CₘH_{f}F_{g} that includes a cyclic alkyl in a chemical structure thereof. “n” is an integer not smaller than 1, “m” is an integer not smaller than 3, and “a”, “b”, “f”, and “g” are each independently an integer not smaller than 0 and satisfy 2n+1=a+b and 2m=f+g.)

In the linear carbonate represented by general formula (1) or general formula (1-1) above, “n” is preferably an integer from 1 to 6, more preferably an integer from 1 to 4, and particularly preferably an integer from 1 to 2. “m” is preferably an integer from 3 to 8, more preferably an integer from 4 to 7, and particularly preferably an integer from 5 to 6.

Among the linear carbonates represented by general formula (1-1) above, dimethyl carbonate (hereinafter, sometimes referred to as “DMC”), diethyl carbonate (hereinafter, sometimes referred to as “DEC”), ethyl methyl carbonate (hereinafter, sometimes referred to as “EMC”), or a material obtained by substituting a part or all of hydrogen atoms of such a linear carbonate with halogen atoms in order to increase oxidation resistance thereof is preferable. Among these, dimethyl carbonate, diethylcarbonate, ethylmethyl carbonate, fluoromethyl methyl carbonate, difluoromethyl methyl carbonate, trifluoromethyl methyl carbonate, bis(fluoromethyl)carbonate, bis(difluoromethyl)carbonate, bis(trifluoromethyl)carbonate, fluoromethyl difluoromethyl carbonate, fluoromethyl trifluoromethyl carbonate, difluoromethyl trifluoromethyl carbonate, 2-fluoroethyl methyl carbonate, 2,2-difluoroethyl methyl carbonate, 2,2,2-trifluoroethyl methyl carbonate, pentafluoroethyl methyl carbonate, ethyl trifluoromethyl carbonate, fluoroethyl ethylcarbonate, trifluoroethyl ethylcarbonate, and bis(2,2,2-trifluoroethyl) carbonate are particularly preferable.

Regarding the linear carbonates described above, a single type may be used in the electrolytic solution or a combination of a plurality of types may be used.

As the heteroelement-containing organic solvent, an organic solvent whose heteroelement is at least one selected from nitrogen, oxygen, sulfur, and a halogen is preferable, and an organic solvent whose heteroelement is oxygen is more preferable. In addition, as the heteroelement-containing organic solvent, an aprotic solvent not having a proton donor group such as NH group, NH₂ group, OH group, and SH group is preferable.

Specific examples of the heteroelement-containing organic solvent include, not to mention linear carbonates represented by general formula (1) above, nitriles such as acetonitrile, propionitrile, acrylonitrile, and malononitrile, ethers such as 1,2-dimethoxyethane, 1,2-diethoxyethane, tetrahydrofuran, 1,2-dioxane, 1,3-dioxane, 1,4-dioxane, 2,2-dimethyl-1,3-dioxolane, 2-methyltetrahydropyran, 2-methyltetrahydrofuran, and crown ethers, carbonates such as ethylene carbonate, propylene carbonate, dimethyl carbonate, diethyl carbonate, and ethyl methyl carbonate, amides such as formamide, N,N-dimethylformamide, N,N-dimethylacetamide, and N-methylpyrrolidone, isocyanates such as isopropyl isocyanate, n-propylisocyanate, and chloromethyl isocyanate, esters such as methyl acetate, ethyl acetate, propyl acetate, methyl propionate, methyl formate, ethyl formate, vinyl acetate, methyl acrylate, and methyl methacrylate, epoxies such as glycidyl methyl ether, epoxy butane, and 2-ethyloxirane, oxazoles such as oxazole, 2-ethyloxazole, oxazoline, and 2-methyl-2-oxazoline, ketones such as acetone, methyl ethyl ketone, and methyl isobutyl ketone, acid anhydrides such as acetic anhydride and propionic anhydride, sulfones such as dimethyl sulfone and sulfolane, sulfoxides such as dimethyl sulfoxide, nitros such as 1-nitropropane and 2-nitropropane, furans such as furan and furfural, cyclic esters such as γ-butyrolactone, γ-valerolactone, and δ-valerolactone, aromatic heterocycles such as thiophene and pyridine, heterocycles such as tetrahydro-4-pyrone, 1-methylpyrrolidine, and N-methylmorpholine, and phosphoric acid esters such as trimethyl phosphate and triethyl phosphate.

The heteroelement-containing organic solvent contained in the electrolytic solution of the present invention contains the linear carbonate represented by general formula (1) above by preferably not less than 80 vol %, more preferably not less than 90 vol %, and further preferably not less than 95 vol %. In addition, the heteroelement-containing organic solvent contained in the electrolytic solution of the present invention contains the linear carbonate represented by general formula (1) above by preferably not less than 80 mole %, more preferably not less than 90 mole %, further preferably not less than 95 mole %.

Examples of a cation of the metal salt in the electrolytic solution of the present invention include alkali metals such as lithium, sodium, and potassium, alkaline earth metals such as beryllium, magnesium, calcium, strontium, and barium, and aluminum. The cation of the metal salt is preferably a metal ion identical to a charge carrier of the battery in which the electrolytic solution is used. For example, when the electrolytic solution of the present invention is to be used as an electrolytic solution for lithium ion secondary batteries, the cation of the metal salt is preferably lithium.

The wording of “optionally substituted with a substituent group” in the chemical structures represented by the above described general formula (2) is to be described. For example, “an alkyl group optionally substituted with a substituent group” refers to an alkyl group in which one or more hydrogen atoms of the alkyl group is substituted with a substituent group, or an alkyl group not including any particular substituent groups.

Examples of the substituent group in the wording of “optionally substituted with a substituent group” include alkyl groups, alkenyl groups, alkynyl groups, cycloalkyl groups, unsaturated cycloalkyl groups, aromatic groups, heterocyclic groups, halogens, OH, SH, CN, SCN, OCN, nitro group, alkoxy groups, unsaturated alkoxy groups, amino group, alkylamino groups, dialkylamino groups, aryloxy groups, acyl groups, alkoxycarbonyl groups, acyloxy groups, aryloxycarbonyl groups, acylamino groups, alkoxycarbonylamino groups, aryloxycarbonylamino groups, sulfonylamino groups, sulfamoyl groups, carbamoyl group, alkylthio groups, arylthio groups, sulfonyl group, sulfinyl group, ureido groups, phosphoric acid amide groups, sulfo group, carboxyl group, hydroxamic acid groups, sulfino group, hydrazino group, imino group, and silyl group, etc. These substituent groups may be further substituted. In addition, when two or more substituent groups exist, the substituent groups may be identical or different from each other.

The chemical structure of the anion of the metal salt is preferably represented by general formula (2-1) below.

(R²³X²²)(R²⁴SO₂)N general formula (2-1)

(R²³ and R²⁴ are each independently CₙHₐF_{b}Cl_{c}Br_{d}Iₑ(CN)_{f}(SCN)_{g}(OCN)ₕ.

“n”, “a”, “b”, “c”, “d”, “e”, “f”, “g”, and “h” are each independently an integer not smaller than 0 and satisfy 2n+1=a+b+c+d+e+f+g+h.

R²³ and R²⁴ optionally bind with each other to form a ring, and in that case, satisfy 2n=a+b+c+d+e+f+g+h.

X²² is selected from SO₂, C═O, C═S, R^{C}P═O, R^{d}P═S, S═O, or Si═O.

R^{c} and R^{d} are each independently selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; OH; SH; CN; SCN; or OCN.

R^{c} and R^{d} each optionally bind with R²³ or R²⁴ to form a ring.)

In the chemical structure represented by general formula (2-1) above, the meaning of the wording of “optionally substituted with a substituent group” is synonymous with that described for the general formula (2) above.

In the chemical structure represented by general formula (2-1) above, “n” is preferably an integer from 0 to 6, more preferably an integer from 0 to 4, and particularly preferably an integer from 0 to 2. In the chemical structure represented by general formula (2-1) above, when R²³ and R²⁴ each optionally bind with each other to form a ring, “n” is preferably an integer from 1 to 8, more preferably an integer from 1 to 7, and particularly preferably an integer from 1 to 3.

The chemical structure of the anion of the metal salt is further preferably represented by general formula (2-2) below.

(R²⁵SO₂)(R²⁶SO₂)N general formula (2-2)

(R²⁵ and R²⁶ are each independently CₙHₐF_{b}Cl_{c}Br_{d}Iₑ.

“n”, “a”, “b”, “c”, “d”, and “e” are each independently an integer not smaller than 0, and satisfy 2n+1=a+b+c+d+e.

R²⁵ and R²⁶ optionally bind with each other to form a ring, and, in that case, satisfy 2n=a+b+c+d+e.)

In the chemical structure represented by general formula (2-2) above, “n” is preferably an integer from 0 to 6, more preferably an integer from 0 to 4, and particularly preferably an integer from 0 to 2. In the chemical structure represented by general formula (2-2) above, when R²⁵ and R²⁶ each optionally bind with each other to form a ring, “n” is preferably an integer from 1 to 8, more preferably an integer from 1 to 7, and particularly preferably an integer from 1 to 3.

In addition, in the chemical structure represented by general formula (2-2) above, those in which “a” is 0, “c” is 0, “d” is 0, and “e” is 0 are each preferable.

The metal salt is particularly preferably (CF₃SO₂)₂NLi (hereinafter, sometimes referred to as “LiTFSA”), (FSO₂)₂NLi (hereinafter, sometimes referred to as “LiFSA”), (C₂F₅SO₂)₂NLi, FSO₂(CF₃SO₂)NLi, (SO₂CF₂CF₂SO₂)NLi, (SO₂CF₂CF₂CF₂SO₂)NLi, FSO₂(CH₃SO₂)NLi, FSO₂(C₂F₅SO₂)NLi, or FSO₂(C₂H₅SO₂)NLi.

As the metal salt of the electrolytic solution of the present invention, one that is obtained by combining appropriate numbers of a cation and an anion described above may be used. Regarding the metal salt in the electrolytic solution of the present invention, a single type may be used, or a combination of two or more types may be used.

The electrolytic solution of the present invention may include another electrolyte usable in an electrolytic solution for power storage devices, other than the metal salt described above. In the electrolytic solution of the present invention, the metal salt is contained by preferably not less than 50 mass %, more preferably not less than 70 mass %, and further preferably not less than 90 mass %, relative to the entire electrolyte contained in the electrolytic solution of the present invention.

The electrolytic solution of the present invention contains a heteroelement-containing organic solvent at a mole ratio of not greater than 1.5 relative to the metal salt. The mole ratio in the present specification means the value obtained by diving the former by the latter, i.e., the value of (the number of moles of the heteroelement-containing organic solvent contained in the electrolytic solution of the present invention/the number of moles of the metal salt contained in the electrolytic solution of the present invention). In the electrolytic solution of the present invention, the mole ratio of the heteroelement-containing organic solvent relative to the metal salt is more preferably in a range of 0.8 to 1.5, and further preferably in a range of 1.0 to 1.5. In conventional electrolytic solutions, the mole ratio of a heteroelement-containing organic solvent relative to an electrolyte is about 10.

Also in another mode of the electrolytic solution of the present invention, the mole ratio of a linear carbonate represented by general formula (1) relative to the metal salt is more preferably in a range of 0.8 to 1.5, and further preferably in a range of 1.0 to 1.5.

In the vibrational spectroscopy spectrum of the electrolytic solution of the present invention, regarding the intensity of a peak derived from a linear carbonate represented by general formula (1) above contained in the electrolytic solution, when the intensity of an original peak of the linear carbonate is defined as Io and the intensity of a peak resulting from shifting of the original peak of the linear carbonate (hereinafter, sometimes referred to as “shift peak”) is defined as Is, Is>Io is satisfied. That is, in a vibrational spectroscopy spectrum chart obtained by subjecting the electrolytic solution of the present invention to vibrational spectroscopy measurement, the relationship between the two peak intensities described above is Is>Io.

In conventional electrolytic solutions, the relationship between Is and Io is Is<Io.

Here, the “original peak of the linear carbonate” means the peak observed at a peak position (wave number) when the vibrational spectroscopy measurement is performed only on the linear carbonate. The value of the intensity Io of the original peak of the linear carbonate and the value of the intensity Is of the shift peak each represent the height or the area from a baseline of each peak in the vibrational spectroscopy spectrum.

In the vibrational spectroscopy spectrum of the electrolytic solution of the present invention, when a plurality of peaks resulting from shifting of the original peak of the linear carbonate exist, the relationship between Is and Io may be determined on the basis of a peak enabling easiest determination of the relationship. In addition, when a plurality of types of the linear carbonate are used in the electrolytic solution of the present invention, a linear carbonate that enables easiest determination of the relationship between Is and Io is selected, and the relationship between Is and Io may be determined on the basis of the peak intensity thereof. In addition, when the peak shift amount is small and peaks before and after the shift overlap with each other to give an appearance like a smooth mountain, the relationship between Is and Io may be determined by performing peak resolution with known means.

The relationship between the two peak intensities in the vibrational spectroscopy spectrum of the electrolytic solution of the present invention preferably satisfies a condition of Is>2×Io, more preferably satisfies a condition of Is>3×Io, still more preferably satisfies a condition of Is>5×Io, and particularly preferably satisfies a condition of Is>7×Io. A most preferable electrolytic solution is one in which the intensity Io of the original peak of the linear carbonate is not observed and the intensity Is of the shift peak is observed in the vibrational spectroscopy spectrum of the electrolytic solution of the present invention. This means that, in the electrolytic solution, all molecules of the linear carbonate contained in the electrolytic solution are completely solvated with the metal salt. The electrolytic solution of the present invention is most preferable in a state where all molecules of the linear carbonate contained in the electrolytic solution are completely solvated with the metal salt (state of Io=0).

In the electrolytic solution of the present invention, the metal salt and the linear carbonate represented by general formula (1) are estimated to interact with each other. Microscopically, the electrolytic solution of the present invention is estimated to contain a stable cluster formed of the metal salt and the linear carbonate, the cluster being formed as a result of formation of a coordinate bond between the metal salt and oxygen in the linear carbonate.

Here, a linear carbonate forming the cluster and a linear carbonate not involved in the formation of the cluster have different environments in which the respective linear carbonates exist. Thus, in the vibrational spectroscopy measurement, a peak derived from the linear carbonate forming the cluster is observed to be shifted toward the high wave number side or the low wave number side with respect to the wave number observed at a peak (i.e., original peak of the linear carbonate) derived from the linear carbonate not involved in the formation of the cluster. That is, the shift peak represents a peak of the linear carbonate forming the cluster.

Examples of the vibrational spectroscopy spectrum include an IR spectrum or a Raman spectrum. Examples of measuring methods of IR spectrum include transmission measuring methods such as Nujol mull method and liquid film method, and reflection measuring methods such as ATR method. Regarding which of the IR spectrum or the Raman spectrum is to be selected, a spectrum enabling easy determination of the relationship between Is and Io may be selected as the vibrational spectroscopy spectrum of the electrolytic solution of the present invention. The vibrational spectroscopy measurement is preferably performed at a condition where the effect of moisture in the atmosphere can be reduced or ignored. For example, performing the IR measurement under a low humidity or zero humidity condition such as in a dry room or a glovebox is preferable, or performing the Raman measurement in a state where the electrolytic solution is kept inside a sealed container is preferable.

Regarding a wave number of a linear carbonate and the attribution thereof, well-known data may be referenced. Examples of the reference include “Raman spectrometry” Spectroscopical Society of Japan measurement method series 17, Hiroo Hamaguchi and Akiko Hirakawa, Japan Scientific Societies Press, pages 231 to 249. In addition, a wave number of a linear carbonate considered to be useful in calculation of Io and Is, and a shift in the wave number when the linear carbonate and the metal salt coordinate with each other are predicted from a calculation using a computer. For example, the calculation may be performed by using Gaussian 09 (Registered trademark, Gaussian, Inc.), and setting the density function to B3LYP and the basis function to 6-311G++(d, p). A person skilled in the art can calculate Io and Is by referring to well-known data and a calculation result from a computer to select a peak of a linear carbonate.

In a vibrational spectroscopy spectrum chart obtained by subjecting the electrolytic solution of the present invention to vibrational spectroscopy measurement, a peak derived from the chemical structure represented by general formula (2) above is sometimes observed to shift to the low wave number side or the high wave number side. Examples of the vibrational spectroscopy spectrum include IR spectrum or Raman spectrum.

Since the electrolytic solution of the present invention contains the metal salt at a high concentration, the cation and the anion forming the metal salt are speculated to strongly interact with each other, so that the metal salt mainly forms a CIP (Contact ion pairs) state or an AGG (aggregate) state. Such a change in the state is observed as a shift of a peak derived from the chemical structure represented by general formula (2) above in the vibrational spectroscopy spectrum chart.

In the electrolytic solution of the present invention, the existence proportion of the metal salt is considered to be high compared to that in conventional electrolytic solutions. Then, in the electrolytic solution of the present invention, the environment in which the metal salt and the organic solvent exist is considered to be different from that in conventional electrolytic solutions. Therefore, in a power storage device such as a secondary battery using the electrolytic solution of the present invention, improvement in metal ion transportation rate in the electrolytic solution, improvement in reaction rate at the interface between an electrode and the electrolytic solution, mitigation of uneven distribution of metal salt concentration of the electrolytic solution caused when the secondary battery undergoes high-rate charging and discharging, improvement in liquid retaining property of the electrolytic solution at an electrode interface, suppression of a so-called liquid run-out state of lacking the electrolytic solution at an electrode interface, increase in the capacity of an electrical double layer, and the like are expected. Furthermore, in the electrolytic solution of the present invention, the vapor pressure of the organic solvent contained in the electrolytic solution becomes low. As a result, volatilization of the organic solvent from the electrolytic solution of the present invention is reduced.

Now, a density d (g/cm³) of the electrolytic solution of the present invention is described. In the present specification, the density defined for the electrolytic solution of the present invention means the density at 30° C. A density d (g/cm³) of the electrolytic solution of the present invention is preferably 1.45≦d and more preferably 1.5≦d.

As reference, densities (g/cm³) of representative heteroelement-containing organic solvents are listed in Table 1.

TABLE 1

| Organic solvent | Density (g/cm³) |
|---|---|
| 1,2-dimethoxyethane | 0.869 |
| diethyl ether | 0.714 |
| diisopropyl ether | 0.724 |
| ethyl acetate | 0.901 |
| acetic anhydride | 1.083 |
| tetrahydrofuran | 0.889 |
| 1,4-dioxane | 1.034 |
| acetone | 0.790 |
| methyl ethyl ketone | 0.805 |
| carbon tetrachloride | 1.594 |
| chloroform | 1.489 |
| dichloromethane | 1.326 |
| 1,2-dichloroethane | 1.252 |
| acetonitrile | 0.782 |
| nitromethane | 1.138 |
| dimethylformamide | 0.949 |
| hexamethylphosphoric triamide | 1.027 |
| triethylamine | 0.728 |
| pyridine | 0.983 |
| dimethyl sulfoxide | 1.100 |
| carbon disulfide | 1.263 |
| ethylene carbonate | 1.321 |
| dimethyl carbonate | 1.07 |
| ethyl methyl carbonate | 1.015 |
| diethyl carbonate | 0.976 |
| sulfolane | 1.261 |

Regarding a viscosity η(mPa·s) of the electrolytic solution of the present invention, a range of 3<η<1000 is preferable, a range of 10<η<600 is more preferable, and a range of 100<η<500 is further preferable.

Ions move in an electrolytic solution easier when an ionic conductivity σ (mS/cm) of the electrolytic solution is higher. Thus, such an electrolytic solution is an excellent electrolytic solution for batteries. The ionic conductivity σ (mS/cm) of the electrolytic solution of the present invention preferably satisfies 1≦σ. Regarding the ionic conductivity σ (mS/cm) of the electrolytic solution of the present invention, if a suitable range including an upper limit is to be shown, a range of 1.0≦σ<100 is preferable, and a range of 1.1≦σ<50 is more preferable.

The electrolytic solution of the present invention contains a cation of the metal salt at a high concentration. Thus, the distance between adjacent cations is extremely small within the electrolytic solution of the present invention. When a cation such as a lithium ion moves between a positive electrode and a negative electrode during charging and discharging of the secondary battery, a cation located closest to an electrode that is a movement destination is firstly supplied to the electrode. Then, to the place where the supplied cation had been located, another cation adjacent to the cation moves. Thus, in the electrolytic solution of the present invention, a domino toppling-like phenomenon is predicted to be occurring in which adjacent cations sequentially change their positions one by one toward an electrode that is a supply target. Because of that, the distance for which a cation moves in the electrolytic solution during charging and discharging is considered to be short, and movement speed of the cation is considered to be high, accordingly. Because of this reason, the electrolytic solution of the present invention is considered to have ion conductivity even at a high viscosity.

In addition, as described later, a secondary battery provided with the electrolytic solution of the present invention is considered to enable reversible and high-speed reaction at the electrode/electrolytic solution interface, since a low resistance SEI coating having a high cation content is formed at the electrode/electrolytic solution interface by use of a substance derived from the metal salt.

The electrolytic solution of the present invention may contain an organic solvent, other than the linear carbonate represented by general formula (1) above. In the electrolytic solution of the present invention, the linear carbonate represented by general formula (1) above is contained, relative to the entire solvent contained in the electrolytic solution of the present invention, by preferably not less than 80 vol %, more preferably not less than 90 vol %, and further preferably not less than 95 vol %. In the electrolytic solution of the present invention, the linear carbonate represented by general formula (1) above is contained, relative to the entire solvent contained in the electrolytic solution of the present invention, by preferably not less than 80 mole %, more preferably not less than 90 mole %, and further preferably not less than 95 mole %.

In some cases, the electrolytic solution of the present invention containing another heteroelement-containing organic solvent, other than the linear carbonate represented by general formula (1) above, has an increased viscosity or a reduced ionic conductivity compared to the electrolytic solution of the present invention not containing another heteroelement-containing organic solvent. Furthermore, in some cases, a secondary battery using the electrolytic solution of the present invention and containing another heteroelement-containing organic solvent, other than the linear carbonate represented by general formula (1) above, has an increased reaction resistance.

The electrolytic solution of the present invention containing an organic solvent formed from the hydrocarbon, other than the linear carbonate represented by general formula (1) above, is expected to have an effect that the viscosity thereof is reduced.

Specific examples of the organic solvent formed from the above hydrocarbon include benzene, toluene, ethyl benzene, o-xylene, m-xylene, p-xylene, 1-methylnaphthalene, hexane, heptane, and cyclohexane.

In addition, to the electrolytic solution of the present invention, a fire-resistant solvent may be added. By adding the fire-resistant solvent to the electrolytic solution of the present invention, safety of the electrolytic solution of the present invention is further enhanced. Examples of the fire-resistant solvent include halogen based solvents such as carbon tetrachloride, tetrachloroethane, and hydrofluoroether, and phosphoric acid derivatives such as trimethyl phosphate and triethyl phosphate.

When the electrolytic solution of the present invention is mixed with a polymer or an inorganic filler to form a mixture, the mixture enables containment of the electrolytic solution to provide a pseudo solid electrolyte. By using the pseudo solid electrolyte as an electrolytic solution of a battery, leakage of the electrolytic solution in the battery is suppressed.

As the polymer, a polymer used in batteries such as lithium ion secondary batteries and a general chemically cross-linked polymer are used. In particular, a polymer capable of turning into a gel by absorbing an electrolytic solution, such as polyvinylidene fluoride and polyhexafluoropropylene, and one obtained by introducing an ion conductive group to a polymer such as polyethylene oxide are suitable.

Specific examples of the polymer include polymethyl acrylate, polymethyl methacrylate, polyethylene oxide, polypropylene oxide, polyacrylonitrile, polyvinylidene fluoride, polyethylene glycol dimethacrylate, polyethylene glycol acrylate, polyglycidol, polytetrafluoroethylene, polyhexafluoropropylene, polysiloxane, polyvinyl acetate, polyvinyl alcohol, polyacrylic acid, polymethacrylic acid, polyitaconic acid, polyfumaric acid, polycrotonic acid, polyangelic acid, polycarboxylic acid such as carboxymethyl cellulose, styrene-butadiene rubbers, nitrile-butadiene rubbers, polystyrene, polycarbonate, unsaturated polyester obtained through copolymerization of maleic anhydride and glycols, polyethylene oxide derivatives having a substituent group, and a copolymer of vinylidene fluoride and hexafluoropropylene. In addition, as the polymer, a copolymer obtained through copolymerization of two or more types of monomers forming the above described specific polymers may be selected.

Polysaccharides are also suitable as the polymer. Specific examples of the polysaccharides include glycogen, cellulose, chitin, agarose, carrageenan, heparin, hyaluronic acid, pectin, amylopectin, xyloglucan, and amylose. In addition, materials containing these polysaccharides may be used as the polymer, and examples of the materials include agar containing polysaccharides such as agarose.

As the inorganic filler, inorganic ceramics such as oxides and nitrides are preferable.

Inorganic ceramics have hydrophilic and hydrophobic functional groups on their surfaces. Thus, a conductive passage may form within the inorganic ceramics when the functional groups attract the electrolytic solution. Furthermore, the inorganic ceramics dispersed in the electrolytic solution form a network among the inorganic ceramics themselves due to the functional groups, and may serve as containment of the electrolytic solution. With such a function by the inorganic ceramics, leakage of the electrolytic solution in the battery is further suitably suppressed. In order to have the inorganic ceramics suitably exert the function described above, the inorganic ceramics having a particle shape are preferable, and those whose particle sizes are nano level are particularly preferable.

Examples of the types of the inorganic ceramics include common alumina, silica, titania, zirconia, and lithium phosphate. In addition, inorganic ceramics that have lithium conductivity themselves are preferable, and specific examples thereof include Li₃N, LiI, LiI—Li₃N—LiOH, LiI—Li₂S—P₂O₅, LiI—Li₂S—P₂S₅, LiI—Li₂S—B₂S₃, Li₂O—B₂S₃, Li₂O—V₂O₃—SiO₂, Li₂O—B₂O₃—P₂O₅, Li₂O—B₂O₃—ZnO, Li₂O—Al₂O₃—TiO₂—SiO₂—P₂O₅, LiTi₂(PO₄)₃, Li-βAl₂O₃, and LiTaO₃.

Glass ceramics may be used as the inorganic filler. Since glass ceramics enables containment of ionic liquids, the same effect is expected for the electrolytic solution of the present invention. Examples of the glass ceramics include compounds represented by xLi₂S-(1−x)P₂S₅ (0<x<1), and those in which one portion of S in the compound is substituted with another element and those in which one portion of P in the compound is substituted with germanium.

Without departing from the gist of the present invention, a known additive may be added to the electrolytic solution of the present invention. Examples of such a known additive include: cyclic carbonates including an unsaturated bond represented by vinylene carbonate (VC), vinylethylene carbonate (VEC), methyl vinylene carbonate (MVC), and ethyl vinylene carbonate (EVC); carbonate compounds represented by fluoro ethylene carbonate, trifluoro propylene carbonate, phenylethylene carbonate, and erythritane carbonate; carboxylic anhydrides represented by succinic anhydride, glutaric anhydride, maleic anhydride, citraconic anhydride, glutaconic anhydride, itaconic anhydride, diglycolic anhydride, cyclohexanedicarboxylic anhydride, cyclopentanetetracarboxylic dianhydride, and phenyl succinic anhydride; lactones represented by γ-butyrolactone, γ-valerolactone, γ-caprolactone, δ-valerolactone, δ-caprolactone, and ε-caprolactone; cyclic ethers represented by 1,4-dioxane; sulfur-containing compounds represented by ethylene sulfite, 1,3-propanesultone, 1,4-butanesultone, methyl methanesulfonate, busulfan, sulfolane, sulfolene, dimethylsulfone, and tetramethylthiuram monosulfide; nitrogen-containing compounds represented by 1-methyl-2-pyrrolidinone, 1-methyl-2-piperidone, 3-methyl-2-oxazolidinone, 1,3-dimethyl-2-imidazolidinone, and N-methylsuccinimide; phosphates represented by monofluorophosphate and difluorophosphate; saturated hydrocarbon compounds represented by heptane, octane, and cycloheptane; and unsaturated hydrocarbon compounds represented by biphenyl, alkyl biphenyl, terphenyl, partially hydrogenated terphenyl, cyclohexylbenzene, t-butylbenzene, t-amyl benzene, diphenyl ether, and dibenzofuran.

The electrolytic solution of the present invention described above is suitably used as an electrolytic solution for power storage devices such as batteries and capacitors. In particular, the electrolytic solution of the present invention is preferably used as electrolytic solutions for secondary batteries, and, among those, preferably used as electrolytic solutions for lithium ion secondary batteries.

Meanwhile, in general, a coating is known to form on the surfaces of the negative electrode and the positive electrode in a secondary battery. This coating is also known as SEI (solid electrolyte interphase), and is formed from reductive/oxidative degradation products, etc. of an electrolytic solution. For example, JP200719027(A) describes such an SEI coating.

The SEI coating on the surface of the negative electrode and the surface of the positive electrode allows a charge carrier such as lithium ions to pass therethrough. In addition, the SEI coating on the surface of the negative electrode/positive electrode is considered to exist between the surface of the negative electrode/positive electrode and the electrolytic solution, and to suppress further reductive/oxidative degradation of the electrolytic solution. Existence of the SEI coating is considered to be essential for a high potential positive electrode operating at 4.5 V or higher and a low potential negative electrode using a graphite- or Si-based negative electrode active material.

If continuous degradation of the electrolytic solution is suppressed due to the existence of the SEI coating, the charge and discharge characteristics of the secondary battery after the charging and discharging cycle are considered to be improved. However, on the other hand, in a conventional secondary battery, the SEI coating on the surfaces of the negative electrode and the positive electrode has not necessarily been considered to contribute to improvement in battery characteristics.

In the electrolytic solution of the present invention, the chemical structure of the metal salt represented by the general formula (2) includes SO₂. When the electrolytic solution of the present invention is used as an electrolytic solution of a secondary battery, an S-and O-containing coating is estimated to be formed on the surface of the positive electrode and/or the negative electrode of the secondary battery as a result of partial degradation of the metal salt through charging and discharging of the secondary battery. The S-and O-containing coating is estimated to have the S═O structure. Since deterioration of the electrodes and the electrolytic solution is suppressed by the electrodes being coated with the coating, durability of the secondary battery is considered to be improved.

In the electrolytic solution of the present invention, a cation and an anion are considered to exist closer to each other when compared to a conventional electrolytic solution, and thus the anion is considered to be more likely to be reduced and degraded on the negative electrode by being under strong electrostatic influence from the cation when compared to a conventional electrolytic solution. In addition, since a large portion of the solvent is in a coordinated state with the metal salt compared to a conventional electrolytic solution, the solvent is considered to be less likely to be oxidized and degraded, and the anion is considered to be relatively more likely to be oxidized and degraded on the positive electrode. Furthermore, in a conventional secondary battery using a conventional electrolytic solution, the SEI coating is formed from a degradation product caused by reductive degradation of a cyclic carbonate such as ethylene carbonate contained in the electrolytic solution. However, as described above, in the electrolytic solution of the present invention in the secondary battery of the present invention, the anion is easy to be reduced and degraded on the negative electrode and oxidized and degraded on the positive electrode, and in addition, the metal salt is contained at a higher concentration than in a conventional electrolytic solution, and thus, the anion concentration in the electrolytic solution is high. Thus, the SEI coating, i.e., the S-and O-containing coating, in the secondary battery of the present invention is considered to contain more degradation product derived from the anion than that of an SEI coating of a conventional secondary battery using a conventional electrolytic solution. In addition, in the secondary battery of the present invention, the SEI coating is formed without using a cyclic carbonate such as ethylene carbonate.

In some cases, the state of the S-and O-containing coating in the secondary battery of the present invention changes associated with charging and discharging. For example, the thickness of the S-and O-containing coating and the proportion of elements in the coating reversibly change sometimes depending on the state of charging and discharging. Thus, a portion that is derived from the degradation product of the anion as described above and is fixed in the coating, and a portion that reversibly increases and decreases associated with charging and discharging are considered to exist in the S-and O-containing coating in the secondary battery of the present invention.

Since the S-and O-containing coating is considered to be derived from the degradation product of the electrolytic solution, a large portion or the entirety of the S-and O-containing coating is considered to be produced during and after the first charging and discharging of the secondary battery. That is, the secondary battery of the present invention has the S-and O-containing coating on the surface of the negative electrode and/or the surface of the positive electrode when being used. Components of the S-and O-containing coating are considered to be sometimes different depending on the composition of the electrode and the components contained in the electrolytic solution. In the S-and O-containing coating, the content proportion of S and O is not limited in particular. Further, components other than S and O and the amount thereof included in the S-and O-containing coating are not limited in particular. Since the S-and O-containing coating is considered to be derived from the anion of the metal salt contained in the electrolytic solution of the present invention, components derived from the anion of the metal salt are preferably contained in an amount greater than that of other components.

The S-and O-containing coating may be formed only on the negative electrode surface or may be formed only on the positive electrode surface. Preferably, the S-and O-containing coating is formed both on the negative electrode surface and the positive electrode surface.

The secondary battery of the present invention includes an S-and O-containing coating on the electrode, and the S-and O-containing coating is considered to have the S═O structure and contain a large amount of the cation. Furthermore, the cation contained in the S-and O-containing coating is considered to be preferentially supplied to the electrode. Thus, since the secondary battery of the present invention has an abundant source of cation near the electrode, transportation rate of the cation is considered to be also improved. As a result, the secondary battery of the present invention is considered to exhibit excellent battery characteristics because of cooperation between the electrolytic solution of the present invention and the S-and O-containing coating on the electrode.

In the following, the lithium ion secondary battery of the present invention provided with the electrolytic solution of the present invention is described.

The lithium ion secondary battery of the present invention includes: a negative electrode having a negative electrode active material capable of occluding and releasing lithium ions; a positive electrode having a positive electrode active material capable of occluding and releasing lithium ions; and the electrolytic solution of the present invention using a lithium salt as the metal salt.

As the negative electrode active material, a material capable of occluding and releasing lithium ions is used. Thus, the material is not limited in particular as long as the material is an elemental substance, an alloy, or a compound capable of occluding and releasing lithium ions. For example, an elemental substance from among Li, group 14 elements such as carbon, silicon, germanium, and tin, group 13 elements such as aluminum and indium, group 12 elements such as zinc and cadmium, group 15 elements such as antimony and bismuth, alkaline earth metals such as magnesium and calcium, and group 11 elements such as silver and gold may be used as the negative electrode active material. When silicon or the like is used as the negative electrode active material, a high capacity active material is obtained since a single silicon atom reacts with multiple lithium atoms. However, a risk of occurrence of a problem regarding a significant expansion and contraction of volume associated with occlusion and release of lithium exists. Thus, in order to mitigate the risk, a substance obtained by combining an elemental substance of silicon or the like with another element such as a transition metal is suitably used as the negative electrode active material. Specific examples of the alloy or the compound include tin-based materials such as Ag—Sn alloys, Cu—Sn alloys, and Co—Sn alloys, carbon based materials such as various graphites, silicon based materials such as SiOₓ (0.3≦x≦1.6) that undergoes disproportionation into the elemental substance silicon and silicon dioxide, and a complex obtained by combining a carbon based material with elemental substance silicon or a silicon based material. In addition, as the negative electrode active material, an oxide such as Nb₂O₅, TiO₂, Li₄Ti₅O₁₂, WO₂, MoO₂, and Fe₂O₃, or a nitride represented by Li₃₋ₓMₓN (M=Co, Ni, Cu) may be used. With regard to the negative electrode active material, one or more types described above may be used.

A more specific example of the negative electrode active material is a graphite whose G/D ratio is not lower than 3.5. The G/D ratio is the ratio of G-band and D-band peaks in a Raman spectrum. In the Raman spectrum of graphite, G-band is observed near 1590 cm⁻¹ and D-band is observed near 1350 cm⁻¹, as peaks, respectively. G-band is derived from a graphite structure and D-band is derived from defects. Thus, having a higher G/D ratio, which is the ratio of G-band and D-band, means the graphite has higher crystallinity with fewer defects. Hereinafter, a graphite whose G/D ratio is not lower than 3.5 is sometimes referred to as a high-crystallinity graphite, and a graphite whose G/D ratio is lower than 3.5 is sometimes referred to as a low-crystallinity graphite.

As such a high-crystallinity graphite, both natural graphites and artificial graphites may be used. When a classification method based on shape is used, flake-like graphites, spheroidal graphites, block-like graphite, earthy graphites, and the like may be used. In addition, coated graphites obtained by coating the surface of a graphite with a carbon material or the like may also be used.

Examples of specific negative electrode active materials include carbon materials whose crystallite size is not larger than 20 nm, and preferably not larger than 5 nm. A larger crystallite size means that the carbon material has atoms arranged periodically and precisely in accordance with a certain rule. On the other hand, a carbon material whose crystallite size is not larger than 20 nm is considered to have atoms being in a state of poor periodicity and poor preciseness in arrangement. For example, when the carbon material is a graphite, the crystallite size becomes not larger than 20 nm when the size of a graphite crystal is not larger than 20 nm or when atoms forming the graphite are arranged irregularly due to distortion, defects, and impurities, etc.

Representative carbon materials whose crystallite size is not larger than 20 nm include hardly graphitizable carbon which is so-called hard carbon, and easily graphitizable carbon which is so-called soft carbon.

In order to measure the crystallite size of the carbon material, an X-ray diffraction method using CuK-alpha radiation as an X-ray source may be used. With the X-ray diffraction method, the crystallite size is calculated using the following Scherrer's equation on the basis of a half width of a diffraction peak detected at a diffraction angle of 2θ=20 degrees to 30 degrees and the diffraction angle.

L=0.94λ/(β cos θ)

where

L: crystallite size

λ: incident X-ray wavelength (1.54 angstrom)

β: half width of peak (radian)

θ: diffraction angle.

Specific examples of the negative electrode active material include materials containing silicon. A more specific example is SiOₓ (0.3≦x≦1.6) disproportionated into two phases of Si phase and silicon oxide phase. The Si phase in SiOₓ is capable of occluding and releasing lithium ions, and changes in volume associated with charging and discharging of the secondary battery. The silicon oxide phase changes less in volume associated with charging and discharging when compared to the Si phase. Thus, SiOₓ as the negative electrode active material achieves higher capacity because of the Si phase, and when included in the silicon oxide phase, suppresses change in volume of the entirety of the negative electrode active material. When “x” becomes smaller than a lower limit value, cycle characteristics of the secondary battery deteriorate since the change in volume during charging and discharging becomes too large due to the ratio of Si becoming excessive. On the other hand, if “x” becomes larger than an upper limit value, energy density is decreased due to the Si ratio being too small. The range of “x” is more preferably 0.5≦x≦1.5, and further preferably 0.7≦x≦1.2.

In SiOₓ described above, an alloying reaction between lithium and silicon in the Si phase is considered to occur during charging and discharging of the lithium ion secondary battery. This alloying reaction is considered to contribute to charging and discharging of the lithium ion secondary battery. Also in the negative electrode active material including tin described later, charging and discharging are considered to occur by an alloying reaction between tin and lithium.

Specific examples of the negative electrode active material include materials containing tin. More specific examples include Sn elemental substance, tin alloys such as Cu—Sn and Co—Sn, amorphous tin oxides, and tin silicon oxides. Examples of the amorphous tin oxides include SnB_{0.4}P_{0.6}O_{3.1}, and examples of the tin silicon oxides include SnSiO₃.

The material containing silicon and the material containing tin described above are each preferably made into a composite with a carbon material to be used as the negative electrode active material. By using those materials as a composite, the structure particularly of silicon and/or tin is stabilized, and durability of the negative electrode is improved. Making a composite mentioned above may be performed by a known method. As the carbon material used in the composite, a graphite, a hard carbon, a soft carbon, etc. may be used. The graphite may be a natural graphite or an artificial graphite.

Specific examples of the negative electrode active material include lithium titanate having a spinel structure such as Li₄₊ₓTi_{5+y}O₁₂ (−1≦x≦4, −1≦y≦1) and lithium titanate having a ramsdellite structure such as Li₂Ti₃O₇.

Specific examples of the negative electrode active material include graphites having a value of long axis/short axis of 1 to 5, and preferably 1 to 3. Here, the long axis means the length of the longest portion of a graphite particle. The short axis means the longest length in directions perpendicular to the long axis. Spheroidal graphites and meso carbon micro beads correspond to the graphite. The spheroidal graphites mean carbon materials which are artificial graphite, natural graphite, easily graphitizable carbon, and hardly graphitizable carbon, for example, and which have spheroidal or substantially spheroidal shapes.

Spheroidal graphite is obtained by grinding graphite into flakes by means of an impact grinder having a relatively small crushing force and by compressing and spheroidizing the flakes. Examples of the impact grinder include a hammer mill and a pin mill. The above operation is preferably performed with the outer-circumference line speed of the hammer or the pin of the mill set at about 50 to 200 m/s. Supply and ejection of graphite with respect to such mills are preferably performed in association with a current of air or the like.

The graphite preferably has a BET specific surface area in a range of 0.5 to 15 m²/g. When the BET specific surface area is too large, side reaction between the graphite and the electrolytic solution is accelerated in some cases. When the BET specific surface area is too small, reaction resistance of the graphite becomes large in some cases.

The negative electrode includes a current collector, and a negative electrode active material layer bound to the surface of the current collector.

The current collector refers to a high-conductivity electron conductor that is chemically inert for continuously sending a flow of current to the electrode during discharging or charging of the lithium ion secondary battery. Examples of the current collector include at least one selected from silver, copper, gold, aluminum, tungsten, cobalt, zinc, nickel, iron, platinum, tin, indium, titanium, ruthenium, tantalum, chromium, or molybdenum, and metal materials such as stainless steel. The current collector may be coated with a known protective layer. One obtained by treating the surface of the current collector with a known method may be used as the current collector.

The current collector takes forms such as a foil, a sheet, a film, a line shape, a bar shape, and a mesh. Thus, as the current collector, for example, metal foils such as copper foil, nickel foil, aluminum foil, and stainless steel foil are suitably used. When the current collector is in the form of a foil, a sheet, or a film, the thickness thereof is preferably in a range of 1 μm to 100 μm.

The negative electrode active material layer includes a negative electrode active material, and, if necessary, a binding agent and/or a conductive additive.

The binding agent serves to adhere the active material and the conductive additive to the surface of the current collector.

As the binding agent, a known binding agent may be used such as a fluorine-containing resin such as polyvinylidene fluoride, polytetrafluoroethylene, or fluororubber, a thermoplastic resin such as polypropylene or polyethylene, an imide based resin such as polyimide or polyamide-imide, an alkoxysilyl group-containing resin, or a styrene butadiene rubber.

In addition, a polymer having a hydrophilic group may be used as the binding agent. Examples of the hydrophilic group of the polymer having a hydrophilic group include carboxyl group, sulfo group, silanol group, amino group, hydroxyl group, and phosphoric acid based group such as phosphoric acid group. Among those described above, a polymer containing a carboxyl group in the molecule thereof, such as polyacrylic acid, carboxymethyl cellulose, and polymethacrylic acid, or a polymer containing a sulfo group such as poly(p-styrenesulfonic acid) is preferable.

A polymer containing a large number of carboxyl groups and/or sulfo groups, such as polyacrylic acid or a copolymer of acrylic acid and vinylsulfonic acid, is water soluble. The polymer containing the hydrophilic group is preferably a water soluble polymer, and is preferably a polymer containing multiple carboxyl groups and/or sulfo groups in a single molecule thereof in terms of the chemical structure.

A polymer containing a carboxyl group in the molecule thereof is produced through, for example, a method of polymerizing an acid monomer or a method of imparting a carboxyl group to a polymer. Examples of the acid monomer include acid monomers having one carboxyl group in respective molecules such as acrylic acid, methacrylic acid, vinylbenzoic acid, crotonic acid, pentenoic acid, angelic acid, and tiglic acid, and acid monomers having two or more carboxyl groups in respective molecules such as itaconic acid, mesaconic acid, citraconic acid, fumaric acid, maleic acid, 2-pentenedioic acid, methylenesuccinic acid, allylmalonic acid, isopropylidene succinic acid, 2,4-hexadienedioic acid, and acetylene dicarboxylic acid.

A copolymer obtained through polymerization of two or more types of acid monomers selected from the acid monomers described above may be used as the binding agent.

For example, as disclosed in JP2013065493(A), a polymer that includes in the molecule thereof an acid anhydride group formed through condensation of carboxyl groups of a copolymer of acrylic acid and itaconic acid is also preferably used as the binding agent. Since the binding agent has a structure derived from a monomer with high acidity by having two or more carboxyl groups in a single molecule thereof, the binding agent is considered to easily trap the lithium ions and the like before a degradation reaction of the electrolytic solution occurs during charging. Furthermore, although the polymer has an increased acidity because the polymer has more carboxyl groups per monomer when compared to polyacrylic acid and polymethacrylic acid, the acidity is not increased too much because a certain amount of carboxyl groups have changed into acid anhydride groups. Therefore, the secondary battery having the negative electrode using the polymer as the binding agent has improved initial efficiency and improved input-output characteristics.

The blending ratio of the binding agent in the negative electrode active material layer in mass ratio is preferably negative electrode active material:binding agent=1:0.005 to 1:0.3. The reason is that when too little of the binding agent is contained, moldability of the electrode deteriorates, whereas too much of the binding agent is contained, energy density of the electrode becomes low.

The conductive additive is added for increasing conductivity of the electrode. Thus, the conductive additive is preferably added optionally when conductivity of the electrode is insufficient, and does not have to be added when conductivity of the electrode is sufficiently good. As the conductive additive, a high-conductivity electron conductor that is chemically inert may be used, and examples thereof include carbonaceous fine particles such as carbon black, graphite, acetylene black, Ketchen black (registered trademark), vapor grown carbon fiber (VGCF), and various metal particles. With regard to the conductive additive described above, a single type by itself, or a combination of two or more types may be added to the active material layer. The blending ratio of the conductive additive in the negative electrode active material layer in mass ratio is preferably negative electrode active material:conductive additive=1:0.01 to 1:0.5. The reason is that when too little of the conductive additive is contained, efficient conducting paths are not formed, whereas when too much of the conductive additive is contained, moldability of the negative electrode active material layer deteriorates and energy density of the electrode becomes low.

The positive electrode used in the lithium ion secondary battery includes a positive electrode active material capable of occluding and releasing lithium ions. The positive electrode includes a current collector and a positive electrode active material layer bound to the surface of the current collector. The positive electrode active material layer includes a positive electrode active material, and, if necessary, a binding agent and/or a conductive additive. The current collector of the positive electrode is not limited in particular as long as the current collector is a metal capable of withstanding a voltage suited for the active material that is used. Examples of the current collector include at least one selected from silver, copper, gold, aluminum, tungsten, cobalt, zinc, nickel, iron, platinum, tin, indium, titanium, ruthenium, tantalum, chromium, and molybdenum, and metal materials such as stainless steel.

When the potential of the positive electrode is set to not lower than 4V using lithium as reference, aluminum is preferably used as the current collector.

Specifically, as the positive electrode current collector, one formed from aluminum or an aluminum alloy is preferably used. Here, aluminum refers to pure aluminum, and an aluminum whose purity is not less than 99.0% is referred to as pure aluminum. An alloy obtained by adding various elements to pure aluminum is referred to as an aluminum alloy. Examples of the aluminum alloy include those that are Al—Cu based, Al—Mn based, Al—Fe based, Al—Si based, Al—Mg based, Al—Mg—Si based, and Al—Zn—Mg based.

In addition, specific examples of aluminum or the aluminum alloy include A1000 series alloys (pure aluminum based) such as JIS A1085, A1N30, etc., A3000 series alloys (Al—Mn based) such as JIS A3003, A3004, etc., and A8000 series alloys (Al—Fe based) such as JIS A8079, A8021, etc.

The current collector may be coated with a known protective layer. One obtained by treating the surface of the current collector with a known method may be used as the current collector.

The current collector takes forms such as a foil, a sheet, a film, a line shape, a bar shape, and a mesh. Thus, as the current collector, for example, metal foils such as copper foil, nickel foil, aluminum foil, and stainless steel foil are suitably used. When the current collector is in the form of a foil, a sheet, or a film, the thickness thereof is preferably in a range of 1 μm to 100 μm.

As the binding agent and the conductive additive for the positive electrode, those described with respect to the negative electrode are used at similar blending ratios.

Examples of the positive electrode active material include layer compounds that are LiₐNi_{b}Co_{c}Mn_{d}DₑO_{f} (0.2≦a≦1.2; b+c+d+e=1; 0≦e<1; D is at least one element selected from Li, Fe, Cr, Cu, Zn, Ca, Mg, S, Si, Na, K, Al, Zr, Ti, P, Ga, Ge, V, Mo, Nb, W, or La; 1.7≦f≦2.1) and Li₂MnO₃. Additional examples of the positive electrode active material include metal oxides having a spinel structure such as LiMn₂O₄, a solid solution formed from a mixture of a metal oxide having a spinel structure and a layer compound, and polyanion based compounds represented by LiMPO₄, LiMVO₄, Li₂MSiO₄ (where “M” is selected from at least one of Co, Ni, Mn, or Fe), or the like. Further additional examples of the positive electrode active material include tavorite based compounds represented by LiMPO₄F (“M” is a transition metal) such as LiFePO₄F and borate based compounds represented by LiMBO₃ (“M” is a transition metal) such as LiFeBO₃. Any metal oxide used as the positive electrode active material may have a basic composition of the composition formulae described above, and those in which a metal element included in the basic composition is substituted with another metal element may also be used. In addition, as the positive electrode active material, one that does not contain a charge carrier (e.g., a lithium ion contributing to the charging and discharging) may also be used. For example, elemental substance sulfur, a compound that is a composite of sulfur and carbon, metal sulfides such as TiS₂, oxides such as V₂O₅ and MnO₂, polyaniline and anthraquinone and compounds containing such aromatics in the chemical structure, conjugate based materials such as conjugate diacetic acid based organic matters, and known other materials may be used. Furthermore, a compound having a stable radical such as nitroxide, nitronyl nitroxide, galvinoxyl, and phenoxyl may be used as the positive electrode active material. When a positive electrode active material not containing a charge carrier such as lithium is to be used, a charge carrier has to be added in advance to the positive electrode and/or the negative electrode using a known method. The charge carrier may be added in an ionic state, or may be added in a nonionic state such as a metal. For example, when the charge carrier is lithium, a lithium foil may be pasted to and integrated with the positive electrode and/or the negative electrode.

Specific examples of the positive electrode active material include LiNi_{0.5}Co_{0.2}Mn_{0.3}O₂, LiNi_{1/3}Co_{1/3}Mn_{1/3}O₂, LiNi_{0.5}Mn_{0.5}O₂, LiNi_{0.75}Co_{0.1}Mn_{0.15}O₂, LiMnO₂, LiNiO₂, and LiCoO₂ having a layered rock salt structure. Another specific example of the positive electrode active material is Li₂MnO₃—LiCoO₂.

Specific examples of the positive electrode active material include LiₓA_{y}Mn_{2-y}O₄ having a spinel structure (“A” is at least one element selected from Ca, Mg, S, Si, Na, K, Al, P, Ga, or Ge, and at least one type of metal element selected from transition metal elements, 0<x≦2.2, 0≦y≦1). More specific examples include LiMn₂O₄, and LiNi_{0.5}Mn_{1.5}O₄.

Specific examples of the positive electrode active material include LiFePO₄, Li₂FeSiO₄, LiCoPO₄, Li₂CoPO₄, Li₂MnPO₄, Li₂MnSiO₄, and Li₂CoPO₄F.

Among these positive electrode active materials, those having a reaction potential not lower than 4.5 V when a Li⁺/Li electrode is used as reference are preferable. Here, “reaction potential” refers to a potential that causes oxidation-reduction reaction of the positive electrode active material through charging and discharging. The reaction potential is based on a Li⁺/Li electrode. Although the reaction potential varies within some range in some cases, “reaction potential” in the present specification refers to the average value of reaction potentials in the range, and when multiple levels of the reaction potentials exist, refers to the average value of the multiple levels of the reaction potentials. Examples of the positive electrode active material whose reaction potential is not lower than 4.5 V when a Li⁺/Li electrode is used as reference include LiNi_{0.5}Mn_{1.5}O₄, LiCoPO₄, Li₂CoPO₄F, Li₂MnO₃—LiMO₂ (where “M” is selected from at least one of Co, Ni, Mn, and Fe), and Li₂MnSiO₄.

In order to form the active material layer on the surface of the current collector, the active material may be applied on the surface of the current collector using a known conventional method such as roll coating method, die coating method, dip coating method, doctor blade method, spray coating method, and curtain coating method. Specifically, an active material layer forming composition containing the active material and, if necessary, the binding agent and the conductive additive, is prepared, and, after adding a suitable solvent to this composition to obtain a paste, the paste is applied on the surface of the current collector and then dried. Examples of the solvent include N-methyl-2-pyrrolidone, methanol, methyl isobutyl ketone, and water. In order to increase electrode density, compression may be performed after drying.

A separator is used in the lithium ion secondary battery, if necessary. The separator is for separating the positive electrode and the negative electrode to allow passage of lithium ions while preventing short circuit due to a contact of both electrodes. As the separator, one that is known may be used. Examples of the separator include porous materials, nonwoven fabrics, and woven fabrics using one or more types of materials having electrical insulation property such as: synthetic resins such as polytetrafluoroethylene, polypropylene, polyethylene, polyimide, polyamide, polyaramide (aromatic polyamide), polyester, and polyacrylonitrile; polysaccharides such as cellulose and amylose; natural polymers such as fibroin, keratin, lignin, and suberin; and ceramics. In addition, the separator may have a multilayer structure.

An example of a method for manufacturing the lithium ion secondary battery of the present invention provided with the electrolytic solution, the positive electrode, and the negative electrode of the present invention is described.

An electrode assembly is formed from the positive electrode, the negative electrode, and, if necessary, the separator interposed therebetween. The electrode assembly may be a laminated type obtained by stacking the positive electrode, the separator, and the negative electrode, or a wound type obtained by winding the positive electrode, the separator, and the negative electrode. The lithium ion secondary battery is preferably formed by respectively connecting, using current collecting leads or the like, the positive electrode current collector to a positive electrode external connection terminal and the negative electrode current collector to a negative electrode external connection terminal, and then adding the electrolytic solution of the present invention to the electrode assembly. In addition, the lithium ion secondary battery of the present invention preferably executes charging and discharging in a voltage range suitable for the types of active materials contained in the electrodes.

The form of the lithium ion secondary battery of the present invention is not limited in particular, and various forms such as a cylindrical type, a square type, a coin type, a laminated type, etc., are used.

The lithium ion secondary battery of the present invention may be mounted on a vehicle. The vehicle may be a vehicle that uses, as all or a part of the source of power, electrical energy obtained from the lithium ion secondary battery, and examples thereof include electric vehicles and hybrid vehicles. When the lithium ion secondary battery is to be mounted on the vehicle, a plurality of the lithium ion secondary batteries may be connected in series to form an assembled battery. Other than the vehicles, examples of instruments on which the lithium ion secondary battery may be mounted include various home appliances, office instruments, and industrial instruments driven by a battery such as personal computers and portable communication devices. In addition, the lithium ion secondary battery of the present invention may be used as power storage devices and power smoothing devices for wind power generation, photovoltaic power generation, hydroelectric power generation, and other power systems, power supply sources for auxiliary machineries and/or power of ships, etc., power supply sources for auxiliary machineries and/or power of aircraft and spacecraft, etc., auxiliary power supply for vehicles that do not use electricity as a source of power, power supply for movable household robots, power supply for system backup, power supply for uninterruptible power supply devices, and power storage devices for temporarily storing power required for charging at charge stations for electric vehicles.

A capacitor of the present invention provided with the electrolytic solution of the present invention may be formed by replacing, with active carbon or the like that is used as a polarized electrode material, a part or all of the negative electrode active material or the positive electrode active material, or a part or all of the negative electrode active material and the positive electrode active material, in the lithium ion secondary battery of the present invention described above. Examples of the capacitor of the present invention include electrical double layer capacitors and hybrid capacitors such as lithium ion capacitors. As the description of the capacitor of the present invention, the description of the lithium ion secondary battery of the present invention above in which “lithium ion secondary battery” is replaced by “capacitor” as appropriate is used.

Although embodiments of the electrolytic solution of the present invention have been described above, the present invention is not limited to the embodiments. Without departing from the gist of the present invention, the present invention can be implemented in various modes with modifications and improvements, etc., that can be made by a person skilled in the art.

### EXAMPLES

In the following, the present invention is specifically described by presenting Examples and Comparative Examples. The present invention is not limited to these Examples.

### Example 1-1

(FSO₂)₂NLi serving as the metal salt was dissolved in dimethyl carbonate, whereby an electrolytic solution of Example 1-1 having (FSO₂)₂NLi at a concentration of 5.5 mol/L was produced. In the electrolytic solution of Example 1-1, the organic solvent is contained at a mole ratio of 1.1 relative to the metal salt.

### Example 1-2

(FSO₂)₂NLi serving as the metal salt was dissolved in dimethyl carbonate, whereby an electrolytic solution of Example 1-2 having (FSO₂)₂NLi at a concentration of 5.0 mol/L was produced. In the electrolytic solution of Example 1-2, the organic solvent is contained at a mole ratio of 1.3 relative to the metal salt.

### Example 2-1

(FSO₂)₂NLi serving as the metal salt was dissolved in ethyl methyl carbonate, whereby an electrolytic solution of Example 2-1 having (FSO₂)₂NLi at a concentration of 5.5 mol/L was produced. In the electrolytic solution of Example 2-1, the organic solvent is contained at a mole ratio of 1.1 relative to the metal salt.

### Comparative Example 1-1

(FSO₂)₂NLi serving as the metal salt was dissolved in dimethyl carbonate, whereby an electrolytic solution of Comparative Example 1-1 having (FSO₂)₂NLi at a concentration of 4.5 mol/L was produced. In the electrolytic solution of Comparative Example 1-1, the organic solvent is contained at a mole ratio of 1.6 relative to the metal salt.

### Comparative Example 1-2

(FSO₂)₂NLi serving as the metal salt was dissolved in dimethyl carbonate, whereby an electrolytic solution of Comparative Example 1-2 having (FSO₂)₂NLi at a concentration of 3.9 mol/L was produced. In the electrolytic solution of Comparative Example 1-2, the organic solvent is contained at a mole ratio of 2 relative to the metal salt.

### Comparative Example 1-3

(FSO₂)₂NLi serving as the metal salt was dissolved in dimethyl carbonate, whereby an electrolytic solution of Comparative Example 1-3 having (FSO₂)₂NLi at a concentration of 3.0 mol/L was produced. In the electrolytic solution of Comparative Example 1-3, the organic solvent is contained at a mole ratio of 3 relative to the metal salt.

### Comparative Example 1-4

(FSO₂)₂NLi serving as the metal salt was dissolved in dimethyl carbonate, whereby an electrolytic solution of Comparative Example 1-4 having (FSO₂)₂NLi at a concentration of 2.0 mol/L was produced. In the electrolytic solution of Comparative Example 1-4, the organic solvent is contained at a mole ratio of 5 relative to the metal salt.

### Comparative Example 1-5

(FSO₂)₂NLi serving as the metal salt was dissolved in dimethyl carbonate, whereby an electrolytic solution of Comparative Example 1-5 having (FSO₂)₂NLi at a concentration of 1.0 mol/L was produced. In the electrolytic solution of Comparative Example 1-5, the organic solvent is contained at a mole ratio of 11 relative to the metal salt.

### Comparative Example 2

LiPF₆ serving as the electrolyte was dissolved in a mixed solvent obtained by mixing dimethyl carbonate and ethylene carbonate at a volume ratio of 1:1, whereby an electrolytic solution of Comparative Example 2 having LiPF₆ at a concentration of 1.0 mol/L was produced. In the electrolytic solution of Comparative Example 2, the organic solvent is contained at a mole ratio of about 10 relative to the electrolyte.

### Comparative Example 3

LiPF₆ serving as the electrolyte was dissolved in a mixed solvent obtained by mixing 3 parts by volume of ethylene carbonate substituted with fluorine and 7 parts by volume of a liquid mixture of a fluorinated linear compound and ethyl methyl carbonate as a low viscosity solvent, whereby an electrolytic solution of Comparative Example 3 having LiPF₆ at a concentration of 1.0 mol/L was produced. In the electrolytic solution of Comparative Example 3, the above-described mixed solvent is contained at a mole ratio of about 10 relative to LiPF₆.

Table 2 shows the list of electrolytic solutions of Examples and Comparative Examples.

TABLE 2

| | | | Number of moles | Concen- |
|---|---|---|---|---|
| | | | of organic | tration |
| | | | solvent/number | of metal |
| | Metal | | of moles of | salt or |
| | salt or | Organic | metal salt or | electrolyte |
| | electrolyte | solvent | electrolyte | (mol/L) |
| Example 1-1 | LiFSA | DMC | 1.1 | 5.5 |
| Example 1-2 | LiFSA | DMC | 1.3 | 5.0 |
| Example 2-1 | LiFSA | EMC | 1.1 | 5.5 |
| Comparative | LiFSA | DMC | 1.6 | 4.5 |
| Example 1-1 | | | | |
| Comparative | LiFSA | DMC | 2 | 3.9 |
| Example 1-2 | | | | |
| Comparative | LiFSA | DMC | 3 | 3.0 |
| Example 1-3 | | | | |
| Comparative | LiFSA | DMC | 5 | 2.0 |
| Example 1-4 | | | | |
| Comparative | LiFSA | DMC | 11 | 1.0 |
| Example 1-5 | | | | |
| Comparative | LiPF₆ | Volume | 10 | 1.0 |
| Example 2 | | ratio of | | |
| | | DMC:EC = 1:1 | | |
| Comparative | LiPF₆ | FEC, EMC, | 10 | 1.0 |
| Example 3 | | F compound | | |

The meanings of abbreviations in Table 2 and thereafter are as follows.

LiFSA: (FSO₂)₂NLi

DMC: dimethyl carbonate

EMC: ethyl methyl carbonate

EC: ethylene carbonate

FEC: ethylene carbonate substituted with fluorine

F compound: fluorinated linear compound

### Evaluation Example 1: Ionic Conductivity

Ionic conductivities of electrolytic solutions of Examples and Comparative Examples were measured under the following conditions. Table 3 shows the results.

Ionic Conductivity Measuring Condition

Under an Ar atmosphere, an electrolytic solution was sealed in a glass cell that had a platinum electrode and whose cell constant was known, and impedance thereof was measured at 30° C., 1 kHz. Ionic conductivity was calculated on the basis of the measurement result of the impedance. As a measurement instrument, Solartron 147055BEC (Solartron Analytical) was used.

TABLE 3

| | | | Number of moles | |
|---|---|---|---|---|
| | | | of organic | |
| | | | solvent/number | Ionic |
| | Metal | | of moles of | conduc- |
| | salt or | Organic | metal salt or | tivity |
| | electrolyte | solvent | electrolyte | (mS/cm) |
| Example 1-1 | LiFSA | DMC | 1.1 | 1.1 |
| Example 1-2 | LiFSA | DMC | 1.3 | 1.7 |
| Comparative | LiFSA | DMC | 1.6 | 2.6 |
| Example 1-1 | | | | |
| Comparative | LiFSA | DMC | 2 | 4.2 |
| Example 1-2 | | | | |
| Comparative | LiFSA | DMC | 3 | 7.8 |
| Example 1-3 | | | | |
| Comparative | LiFSA | DMC | 5 | 11.8 |
| Example 1-4 | | | | |
| Comparative | LiFSA | DMC | 11 | 9.2 |
| Example 1-5 | | | | |
| Comparative | LiPF₆ | Volume | 10 | 13.1 |
| Example 2 | | ratio of | | |
| | | DMC:EC = 1:1 | | |

The electrolytic solutions of Examples all showed ion conductivity of not less than 1 mS/cm. Thus, the electrolytic solutions of the present invention are all understood to function as electrolytic solutions of various types of power storage devices.

### Evaluation Example 2: Density

Densities at 30° C. of electrolytic solutions of Examples and Comparative Examples were measured. Table 4 shows the results.

TABLE 4

| | | | Number of moles | |
|---|---|---|---|---|
| | | | of organic | |
| | | | solvent/number | |
| | Metal | | of moles of | |
| | salt or | Organic | metal salt or | Density |
| | electrolyte | solvent | electrolyte | (g/cm³) |
| Example 1-1 | LiFSA | DMC | 1.1 | 1.58 |
| Example 1-2 | LiFSA | DMC | 1.3 | 1.54 |
| Comparative | LiFSA | DMC | 1.6 | 1.49 |
| Example 1-1 | | | | |
| Comparative | LiFSA | DMC | 2 | 1.44 |
| Example 1-2 | | | | |
| Comparative | LiFSA | DMC | 3 | 1.36 |
| Example 1-3 | | | | |
| Comparative | LiFSA | DMC | 5 | 1.27 |
| Example 1-4 | | | | |
| Comparative | LiFSA | DMC | 11 | 1.17 |
| Example 1-5 | | | | |
| Comparative | LiPF₆ | Volume | 10 | 1.29 |
| Example 2 | | ratio of | | |
| | | DMC:EC = 1:1 | | |

### Evaluation Example 3: Viscosity

Viscosities of electrolytic solutions of Examples and Comparative Examples were measured under the following conditions. Table 5 shows the results.

Viscosity Measuring Condition

Under an Ar atmosphere, an electrolytic solution was sealed in a test cell, and viscosity was measured under a condition of 30° C. by using a falling ball viscometer (Lovis 2000 M manufactured by Anton Paar GmbH).

TABLE 5

| | | | Number of moles | |
|---|---|---|---|---|
| | | | of organic | |
| | | | solvent/number | |
| | Metal | | of moles of | |
| | salt or | Organic | metal salt or | Viscosity |
| | electrolyte | solvent | electrolyte | (mPa · s) |
| Example 1-1 | LiFSA | DMC | 1.1 | 449 |
| Example 1-2 | LiFSA | DMC | 1.3 | 202 |
| Comparative | LiFSA | DMC | 1.6 | 105 |
| Example 1-1 | | | | |
| Comparative | LiFSA | DMC | 2 | 50.9 |
| Example 1-2 | | | | |
| Comparative | LiFSA | DMC | 3 | 17.6 |
| Example 1-3 | | | | |
| Comparative | LiFSA | DMC | 5 | 5.4 |
| Example 1-4 | | | | |
| Comparative | LiFSA | DMC | 11 | 1.8 |
| Example 1-5 | | | | |
| Comparative | LiPF₆ | Volume | 10 | 3.1 |
| Example 2 | | ratio of | | |
| | | DMC:EC = 1:1 | | |

The viscosities of the electrolytic solutions of Examples were understood to be significantly high compared to the viscosities of the electrolytic solutions of Comparative Examples. Thus, power storage devices using the electrolytic solution of the present invention suppress leakage of the electrolytic solution even if the power storage devices get broken.

### Evaluation Example 4: Raman Spectrum Measurement

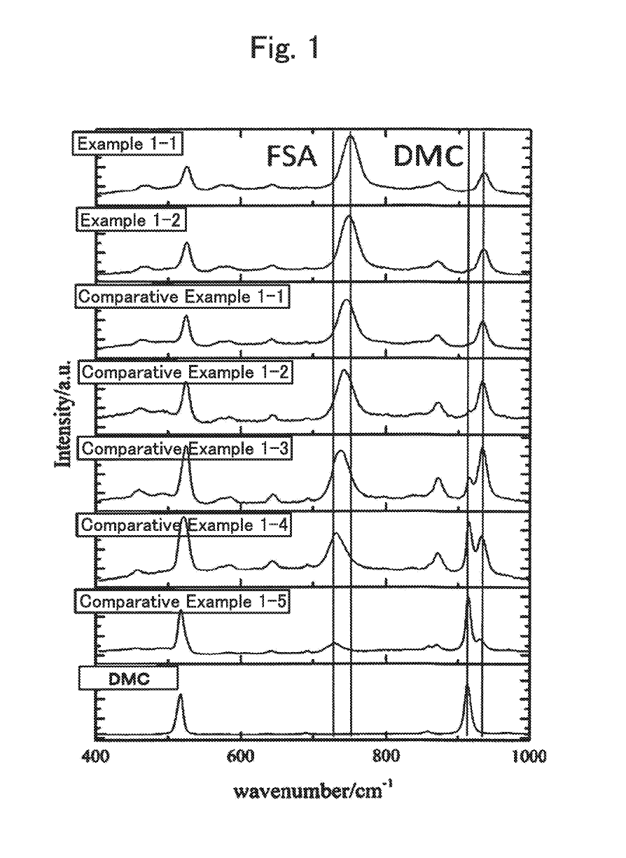

Raman spectrum measurement was performed on the electrolytic solutions of Example 1-1, Example 1-2, and Comparative Examples 1-1 to 1-5, and on DMC, under the following conditions. FIG. 1 shows a Raman spectrum in which a portion derived from DMC of each electrolytic solution and an anion portion of the metal salt, i.e., a peak derived from (FSO₂)₂N, were observed. In FIG. 1, the horizontal axis represents wave number (cm⁻¹), and the vertical axis represents scattering intensity. FSA indicated in FIG. 1 is the abbreviation of (FSO₂)₂N.

Raman Spectrum Measuring Condition

Apparatus: Laser Raman spectrometer (NRS series, JASCO Corp.)

Laser wavelength: 532 nm

Laser power: 50 mW

Exposure time: 20 to 40 seconds

Integration times: 3

Each electrolytic solution was sealed in a quartz cell under an inert gas atmosphere, and was subjected to the measurement.

In 700 to 800 cm⁻¹ of the Raman spectrum of the electrolytic solution of each of Example 1-1, Example 1-2, Comparative Examples 1-1 to 1-5 shown in FIG. 1, a characteristic peak derived from (FSO₂)₂N of LiFSA was observed. With reference to FIG. 1, the peak is understood to shift toward the high wave number side in association with decrease in the mole ratio of DMC/LiFSA, i.e., in association with increase in the concentration of LiFSA. Here, (FSO₂)₂N corresponding to the anion of a salt is speculated to enter a state of interacting with Li in accordance with increase in the concentration of the electrolytic solution. In other words, Li and the anion are speculated to mainly form an SSIP (solvent-separated ion pairs) state when the concentration is low, and are speculated to mainly form a CIP (Contact ion pairs) state and an AGG (aggregate) state in accordance with increase in the concentration. Thus, change in such states is considered to have been observed as the peak shift in the Raman spectrum.

In addition, near 900 to 950 cm⁻¹ of the Raman spectrum of each of the electrolytic solutions of Example 1-1, Example 1-2, and Comparative Examples 1-1 to 1-5 and of DMC shown in FIG. 1, a characteristic peak derived from DMC was observed. From the Raman spectrum of each of Comparative Example 1-2 to Comparative Example 1-5 shown in FIG. 1, the peak derived from DMC is understood to be observed, separated at an original peak wave number and at a shifted peak wave number. In addition, the relationship between the intensity Io of the original peak and the intensity Is of the peak resulting from shifting of the original peak is Io>Is in the case of the electrolytic solution of Comparative Example 1-5 where DMC/LiFSA=11, and also is Io>Is in the case of electrolytic solution of Comparative Example 1-4 where DMC/LiFSA=5. On the other hand, in the case of the electrolytic solution of Comparative Example 1-3 where DMC/LiFSA=3, the above relationship is Io<Is, and also is Io<Is in the case of the electrolytic solution of Comparative Example 1-2 where DMC/LiFSA=2. That is, Io is considered to decrease and Is is considered to increase, in accordance with decrease in the value of DMC/LiFSA. In the electrolytic solutions of Example 1-1, Example 1-2, and Comparative Example 1-1, no Io was confirmed, and only Is was observed.

With respect to the electrolytic solutions of Example 1-1 and Example 1-2, substantially all molecules of the linear carbonate contained in the electrolytic solution were confirmed to be solvated with the metal salt.

### Example A

A half-cell using the electrolytic solution of Example 1-2 was produced in the following manner.

An aluminum foil (JIS A1000 series) having a diameter of 13.82 mm, an area of 1.5 cm², and a thickness of 20 μm was used as the working electrode, and metal Li was used as the counter electrode. As the separator, a microporous separator made from polypropylene and having a thickness of 30 μm was used.

The working electrode, the counter electrode, the separator, and the electrolytic solution of Example 1-2 were housed in a battery case (CR2032 type coin cell case manufactured by Hohsen Corp.), to form a half-cell. This was used as a half-cell of Example A.

### Comparative Example A

A half-cell of Comparative Example A was created similarly to the half-cell of Example A except for using the electrolytic solution of Comparative Example 1-1.

### Comparative Example B

A half-cell of Comparative Example B was created similarly to the half-cell of Example A except for using the electrolytic solution of Comparative Example 1-2.

### Comparative Example C

A half-cell of Comparative Example C was created similarly to the half-cell of Example A except for using the electrolytic solution of Comparative Example 1-3.

### Comparative Example D

A half-cell of Comparative Example D was created similarly to the half-cell of Example A except for using the electrolytic solution of Comparative Example 1-4.

### Comparative Example E

A half-cell of Comparative Example E was created similarly to the half-cell of Example A except for using the electrolytic solution of Comparative Example 1-5.

### Evaluation Example A: Linear Sweep Voltammetry Evaluation at Working Electrode Al

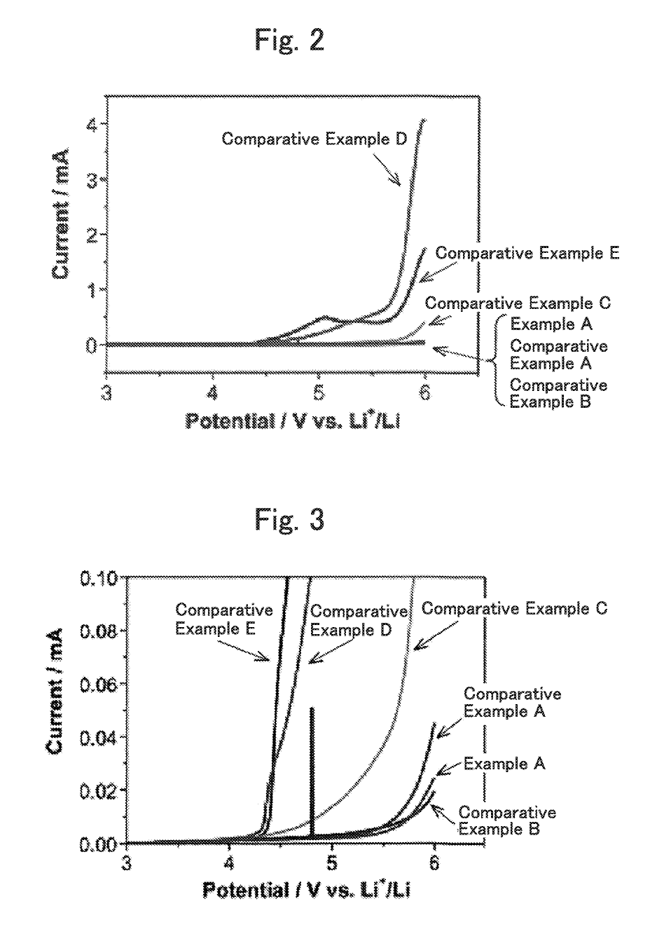

Linear sweep voltammetry was performed in which the half-cell of each of Example A and Comparative Examples A to E was subjected to continuous potential change under a condition of 1 mV/s in a range of potential 3.0 V to 6.0 V relative to Li, and the relationship between the potential and the response current was observed. FIG. 2 and FIG. 3 each show a graph of potential and response current. FIG. 3 shows the graph of FIG. 2 in an enlarged manner.

FIG. 2 and FIG. 3 reveal that: in the half-cell of each of Comparative Example C, Comparative Example D, and Comparative Example E, current started to flow at a potential near 4.3 V; and the current increased when the potential became higher. These currents are each estimated to be a current resulting from oxidation of Al, generated through corrosion of aluminum of the working electrode.

FIG. 2 and FIG. 3 reveal that, in the half-cell of Example A, Comparative Example A, and Comparative Example B, substantially no current flowed from 3.0 V to 5.5 V. FIG. 3 reveals that the potential at which current starts to flow is highest in the half-cell of Example A. That is, among the half-cells of Example A and Comparative Examples A to E, the half-cell of Example A is considered to be most excellent in terms of potential durability.

From the results of the linear sweep voltammetry evaluation, corrosiveness and oxidative degrading property of the electrolytic solution of Example 1-2 with respect to aluminum are considered to be low even under a condition of high potential exceeding 4.5 V. That is, the electrolytic solution of Example 1-2 is considered to be a suitable electrolytic solution for power storage devices using aluminum for a current collector and the like.

### Example B

A half-cell using the electrolytic solution of Example 1-2 was produced in the following manner.

80 parts by mass of LiNi_{0.5}Mn_{1.5}O₄ having a spinel structure and serving as the active material, 5 parts by mass of polyvinylidene fluoride serving as the binding agent, and 15 parts by mass of acetylene black serving as the conductive additive were mixed. This mixture was dispersed in a proper amount of N-methyl-2-pyrrolidone to create a slurry. As the current collector, an aluminum foil having a thickness of 20 μm was prepared. The slurry was applied in a film form on the surface of the aluminum foil by using a doctor blade. The aluminum foil on which the slurry was applied was dried to remove N-methyl-2-pyrrolidone, and then the aluminum foil was pressed to obtain a joined object. The obtained joined object was heated and dried in a vacuum dryer for 6 hours at 120° C. to obtain an aluminum foil having an active material layer formed thereon. This was used as a working electrode.

Metal Li was Used as the Counter Electrode.

The working electrode, the counter electrode, a 30 μm-thick microporous separator interposed therebetween and made from polypropylene, and the electrolytic solution of Example 1-2 were housed in a battery case (CR2032 type coin cell case manufactured by Hohsen Corp.), to form a half-cell. This was used as a half-cell of Example B.

### Comparative Example F

A half-cell of Comparative Example F was produced using a method similar to that in Example B except for using the electrolytic solution of Comparative Example 1-1 as the electrolytic solution.

### Comparative Example G

A half-cell of Comparative Example G was produced using a method similar to that in Example B except for using the electrolytic solution of Comparative Example 2 as the electrolytic solution.

### Evaluation Example B: Capacity Retention Rate of Half-Cell

The capacity retention rate of the half-cell of each of Example B, Comparative Example F, and Comparative Example G was tested by using the following method.

For each half-cell, a charging and discharging cycle was repeated at room temperature in a range of 3.5 V to 4.9 V (vs. Li reference) at 0.2 C, to measure the discharge capacity at each cycle. For each half-cell, the capacity retention rate (%) was calculated by the formula below. In the description here, the counter electrode is regarded as the negative electrode, and the working electrode is regarded as the positive electrode. Table 6 shows the results.

Capacity retention rate (%)=100×(discharge capacity at 30-th cycle)/(discharge capacity at 1st cycle)

TABLE 6

| | | Organic solvent of | |
|---|---|---|---|
| | | electrolytic solution | |
| | | Metal salt or electrolyte | |
| | | Number of moles of organic | Capacity |
| | Electrolytic | solvent/number of moles of | retention |
| Half-cell | solution | metal salt or electrolyte | rate (%) |
| Example B | Example 1-2 | DMC | 100 |
| | | LiFSA | |
| | | 1.3 | |
| Comparative | Comparative | DMC | 80 |
| Example F | Example 1-1 | LiFSA | |
| | | 1.6 | |
| Comparative | Comparative | DMC and EC (volume ratio 1:1) | 96 |
| Example G | Example 2 | LiPF₆ | |
| | | 10 | |

From the results shown in Table 6, the half-cell of Example B is understood to show a significantly excellent capacity retention rate. A secondary battery provided with LiNi_{0.5}Mn_{1.5}O₄ having a spinel structure as the active material and the electrolytic solution of the present invention was confirmed to show an excellent capacity retention rate.

### Example C

A half-cell using the electrolytic solution of Example 1-2 was produced in the following manner.

80 parts by mass of LiNi_{0.5}Mn_{1.5}O₄ having a spinel structure and serving as the active material, 10 parts by mass of polyvinylidene fluoride serving as the binding agent, and 10 parts by mass of acetylene black serving as the conductive additive were mixed. This mixture was dispersed in a proper amount of N-methyl-2-pyrrolidone to create a slurry. As the current collector, an aluminum foil having a thickness of 20 μm was prepared. The slurry was applied in a film form on the surface of the aluminum foil by using a doctor blade. The aluminum foil on which the slurry was applied was dried to remove N-methyl-2-pyrrolidone, and then the aluminum foil was pressed to obtain a joined object. The obtained joined object was heated and dried in a vacuum dryer for 6 hours at 120° C. to obtain an aluminum foil having an active material layer formed thereon. This was used as a working electrode.

Metal Li was Used as the Counter Electrode.

The working electrode, the counter electrode, a 400 μm-thick separator interposed therebetween and made from glass fiber, and the electrolytic solution of Example 1-2 were housed in a battery case (CR2032 type coin cell case manufactured by Hohsen Corp.), to form a half-cell. This was used as a half-cell of Example C.

### Comparative Example H

A half-cell of Comparative Example H was produced using a method similar to that in Example C except for using the electrolytic solution of Comparative Example 1-1 as the electrolytic solution.

### Comparative Example I

A half-cell of Comparative Example I was produced using a method similar to that in Example C except for using the electrolytic solution of Comparative Example 2 as the electrolytic solution.

### Evaluation Example C: Rate Capacity Test

The rate capacity of the half-cell of each of Example C, Comparative Example H, and Comparative Example I was tested using the following method.

A charging and discharging cycle test was performed in which, with respect to each half-cell, charging from 3.5 V to 4.9 V and discharging from 4.9 V to 3.5 V were performed at room temperature in the rate order of 0.1 C, 0.2 C, 0.5 C, 1 C, 2 C, 5 C, 10 C, 15 C, 20 C, and 0.1 C, three times for each rate. Table 7 shows results obtained by calculating the ratio of the discharge capacity at the second time at 5 C rate relative to the discharge capacity at the second time at the initial 0.1 C rate. In the description here, the counter electrode is regarded as the negative electrode, and the working electrode is regarded as the positive electrode. 1 C means the current value required for fully charging or fully discharging the battery in 1 hour with a constant current.

TABLE 7

| | | Organic solvent of | |
|---|---|---|---|
| | | electrolytic solution | |
| | | Metal salt or electrolyte | |
| | | Number of moles of organic | 5 C discharge |
| | Electrolytic | solvent/number of moles of | capacity/0.1 C |
| Half-cell | solution | metal salt or electrolyte | discharge capacity |
| Example C | Example 1-2 | DMC | 55 |
| | | LiFSA | |
| | | 1.3 | |
| Comparative | Comparative | DMC | 24 |
| Example H | Example 1-1 | LiFSA | |
| | | 1.6 | |
| Comparative | Comparative | DMC and EC (volume ratio 1:1) | 39 |
| Example I | Example 2 | LiPF₆ | |
| | | 10 | |

With reference to the results shown in Table 7, the half-cell of Example C has suppressed decrease in the capacity at 5 C rate compared to the half-cell of each of the Comparative Examples, and showed an excellent rate characteristic. A secondary battery provided with LiNi_{0.5}Mn_{1.5}O₄ having a spinel structure as the active material and the electrolytic solution of the present invention was confirmed to show an excellent rate characteristic. In addition, in the half-cell of Example C, the ratio of the charge capacity at the second time at the last 0.1 C rate relative to the charge capacity at the second time at the initial 0.1 C rate was 100%. With respect to the secondary battery of the present invention, the capacity was confirmed to be sufficiently retained even when charging and discharging are performed after rapid charging and discharging.

### Example D

A half-cell using the electrolytic solution of Example 1-2 was produced in the following manner.

90 parts by mass of a graphite having a mean particle diameter of 10 μm and serving as the active material was mixed with 10 parts by mass of polyvinylidene fluoride serving as the binding agent. This mixture was dispersed in a proper amount of N-methyl-2-pyrrolidone to create a slurry. As the current collector, a copper foil having a thickness of 20 μm was prepared. The slurry was applied in a film form on the surface of the copper foil by using a doctor blade. The copper foil on which the slurry was applied was dried to remove N-methyl-2-pyrrolidone, and then the copper foil was pressed to obtain a joined object. The obtained joined object was heated and dried in a vacuum dryer for 6 hours at 120° C. to obtain a copper foil having an active material layer formed thereon. This was used as a working electrode. As the graphite, an SNO grade graphite from SEC CARBON LIMITED was used.

Metal Li was Used as the Counter Electrode.

The working electrode, the counter electrode, a 30 μm-thick microporous separator interposed therebetween and made from polypropylene, and the electrolytic solution of Example 1-2 were housed in a battery case (CR2032 type coin cell case manufactured by Hohsen Corp.), to form a half-cell. This was used as a half-cell of Example D.

### Comparative Example J

A half-cell of Comparative Example J was created similarly to the half-cell of Example D except for using the electrolytic solution of Comparative Example 1-1.

### Comparative Example K

A half-cell of Comparative Example K was created similarly to the half-cell of Example D except for using the electrolytic solution of Comparative Example 1-2.

### Comparative Example L

A half-cell of Comparative Example L was created similarly to the half-cell of Example D except for using the electrolytic solution of Comparative Example 1-3.

### Comparative Example M

A half-cell of Comparative Example M was created similarly to the half-cell of Example D except for using the electrolytic solution of Comparative Example 1-4.

### Comparative Example N

A half-cell of Comparative Example N was created similarly to the half-cell of Example D except for using the electrolytic solution of Comparative Example 1-5.

### Comparative Example O

A half-cell of Comparative Example O was created similarly to the half-cell of Example D except for using the electrolytic solution of Comparative Example 2.

### Evaluation Example D: Confirmation of Irreversible Capacity

With respect to the half-cell of each of Example D and Comparative Examples J to O, charging and discharging were performed in a range of 2.5 V to 0.1 V at 0.1 C at 25° C. to observe the charging curve and the discharging curve. In addition, the first charge capacity and the first discharge capacity of each half-cell was measured, and further, the first coulombic efficiency (%) was calculated by the calculation formula below. Table 8 shows the results.

First coulombic efficiency (%)=100×(first discharge capacity)/(first charge capacity)

In the description here, the counter electrode is regarded as the positive electrode, and the working electrode is regarded as the negative electrode.

The charging curve of the half-cell of Example D provided with the electrolytic solution of the present invention showed stepwise potential change not greater than 0.25 V corresponding to the stage structure of the Li-graphite intercalation compound, similarly to the charging curve of the half-cell of Comparative Example O provided with a conventional electrolytic solution. In addition, Table 8 reveals that the half-cell of Example D has a higher coulombic efficiency than the coulombic efficiency of the half-cell of each of Comparative Examples J to O, and has a smaller first irreversible capacity. The half-cell of Example D is also understood to enable suitable reversible charging and discharging.

TABLE 8

| | | Organic solvent of | | | |
|---|---|---|---|---|---|
| | | electrolytic solution | | | |
| | | Metal salt or electrolyte | First | First | First |
| | | Number of moles of organic | charge | discharge | coulombic |
| | Electrolytic | solvent/number of moles of | capacity | capacity | efficiency |
| Half-cell | solution | metal salt or electrolyte | (mAh/g) | (mAh/g) | (%) |
| Example D | Example 1-2 | DMC | 428 | 357 | 83.4 |
| | | LiFSA | | | |
| | | 1.3 | | | |
| Comparative | Comparative | DMC | 430 | 354 | 82.3 |
| Example J | Example 1-1 | LiFSA | | | |
| | | 1.6 | | | |
| Comparative | Comparative | DMC | 445 | 360 | 80.9 |
| Example K | Example 1-2 | LiFSA | | | |
| | | 2 | | | |
| Comparative | Comparative | DMC | 466 | 368 | 79.0 |
| Example L | Example 1-3 | LiFSA | | | |
| | | 3 | | | |
| Comparative | Comparative | DMC | 488 | 369 | 75.6 |
| Example M | Example 1-4 | LiFSA | | | |
| | | 5 | | | |
| Comparative | Comparative | DMC | 477 | 359 | 75.3 |
| Example N | Example 1-5 | LiFSA | | | |
| | | 11 | | | |
| Comparative | Comparative | DMC and EC (volume ratio 1:1) | 436 | 363 | 83.3 |
| Example O | Example 2 | LiPF₆ | | | |
| | | 10 | | | |

Meanwhile, a lithium ion secondary battery having a conventional graphite negative electrode is known to enable reversible charging and discharging by being provided with the EC-containing electrolytic solution as shown in Comparative Example 2 and thereby forming an SEI coating on the graphite negative electrode. However, although the half-cell of Example D above is provided with an EC-absent electrolytic solution, the half-cell of Example D above was shown to enable suitable reversible charging and discharging, similarly to that provided with the EC-containing electrolytic solution. The reason for this is considered as follows. A good SEI coating mainly composed of a substance derived from the metal salt is formed on the graphite negative electrode due to both of: the mole ratio of the heteroelement-containing organic solvent relative to the metal salt being significantly low; and a specific metal salt being selected, both being the characteristics of the electrolytic solution of the present invention.

### Example I

A lithium ion secondary battery using the electrolytic solution of Example 1-1 was produced in the following manner.

89 parts by mass of LiNi_{0.5}Mn_{0.5}O₄ having a spinel structure and serving as the positive electrode active material, 8 parts by mass of acetylene black serving as the conductive additive, and 3 parts by mass of polyvinylidene fluoride serving as the binding agent were mixed. This mixture was dispersed in a proper amount of N-methyl-2-pyrrolidone to create a slurry. As the positive electrode current collector, an aluminum foil having a thickness of 20 μm was prepared. The slurry was applied in a film form on the surface of the aluminum foil by using a doctor blade. The aluminum foil on which the slurry was applied was dried for 20 minutes at 80° C. to remove N-methyl-2-pyrrolidone through volatilization. Then, the aluminum foil was pressed to obtain a joined object. The obtained joined object was heated and dried in a vacuum dryer for 6 hours at 120° C. to obtain an aluminum foil having a positive electrode active material layer formed thereon. This was used as the positive electrode.

98 parts by mass of graphite serving as the negative electrode active material, and 1 part by mass of styrene-butadiene-rubber and 1 part by mass of carboxymethyl cellulose, which both served as the binding agent, were mixed. This mixture was dispersed in a proper amount of ion exchanged water to create a slurry. As the negative electrode current collector, a copper foil having a thickness of 20 μm was prepared. The slurry was applied in a film form on the surface of the copper foil by using a doctor blade. The copper foil on which the slurry was applied was dried to remove water, and then, the copper foil was pressed to obtain a joined object. The obtained joined object was heated and dried in a vacuum dryer for 6 hours at 100° C. to obtain a copper foil having a negative electrode active material layer formed thereon. This was used as the negative electrode.

As the separator, a nonwoven fabric made from cellulose and having a thickness of 20 μm was prepared.

An electrode assembly was formed by sandwiching the separator between the positive electrode and the negative electrode. The electrode assembly was covered with a set of two sheets of a laminate film. The laminate film was formed into a bag-like shape by having three sides thereof sealed, and the electrolytic solution of Example 1-1 was poured into the laminate film. Four sides were sealed airtight by sealing the remaining one side to obtain a lithium ion secondary battery in which the electrode assembly and the electrolytic solution were sealed. This battery was used as the lithium ion secondary battery of Example I.

### Example II

A lithium ion secondary battery of Example II was created similarly to the lithium ion secondary battery of Example I except for using the electrolytic solution of Example 2-1.

### Comparative Example I

A lithium ion secondary battery of Comparative Example I was created similarly to the lithium ion secondary battery of Example I except for using the electrolytic solution of Comparative Example 3.

### Evaluation Example I: Capacity Retention Rate of Lithium Ion Secondary Battery

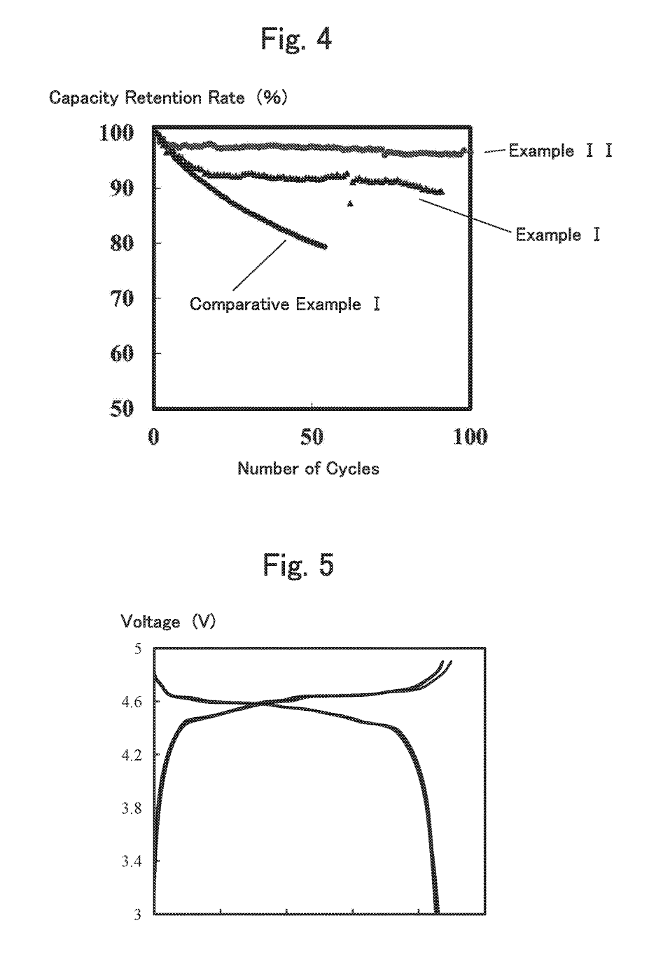

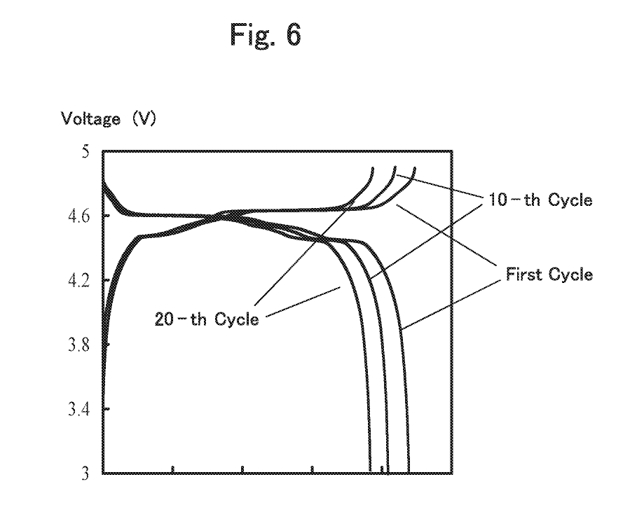

With respect to the lithium ion secondary battery of Examples I and II and Comparative Example I, the capacity retention rate was evaluated by using the following method. For each lithium ion secondary battery, charging and discharging were repeated in a range of 3.0 V to 4.9 V (vs. Li reference) at 0.1 C at room temperature. The discharge capacity during the first charging and discharging and the discharge capacity at each cycle were measured. Then, the capacity of each lithium ion secondary battery during the first discharging was defined as 100%, the capacity retention rate (%) of each lithium ion secondary battery at a specific cycle was calculated. Table 9 and FIG. 4 show the results. In addition, with respect to the lithium ion secondary battery of Example II, FIG. 5 shows the charging and discharging curves of the first cycle, the 10-th cycle, the 20-th cycle, and the 45-th cycle. With respect to the lithium ion secondary battery of Comparative Example I, FIG. 6 shows the charging and discharging curves of the first cycle, the 10-th cycle, and the 20-th cycle.

TABLE 9

| | | Organic solvent of | |
|---|---|---|---|
| | | electrolytic solution | |
| | | Metal salt or electrolyte | |
| | | Number of moles of organic | Capacity |
| | Electrolytic | solvent/number of moles of | retention |
| | solution | metal salt or electrolyte | rate |
| Example I | Example | DMC | 50-th |
| | 1-1 | LiFSA | cycle: 92% |
| | | 1.1 | 80-th |
| | | | cycle: 91% |
| Example II | Example | EMC | 50-th |
| | 2-1 | LiFSA | cycle: 98% |
| | | 1.1 | 100-th |
| | | | cycle: 97% |
| Comparative | Comparative | FEC, EMC, F compound | 50-th |
| Example I | Example 3 | LiPF₆ | cycle: 80% |
| | | 10 | |

From Table 9 and FIG. 4, secondary batteries provided with the electrolytic solution of the present invention were confirmed to suitably retain the capacity even in the charging and discharging cycle at a high potential of 4.9 V. From the results of Example I and Example II, increase in the carbon number of R¹⁰ and/or R¹¹ in general formula (1) in the electrolytic solution of the present invention was suggested to contribute to making suitable the capacity retention rate of the secondary battery.

FIG. 5 shows that the charging and discharging curves of the lithium ion secondary battery of Example II substantially match the first charging and discharging curves even after a plurality of cycles, and reveals that excessive charge capacity as caused by degradation of the electrolytic solution was not observed. This result is considered to indicate that the electrolytic solution of the present invention was stable even under high potentials, and that no major polarization occurred. The electrolytic solution of the present invention was confirmed to be an electrolytic solution suitable for batteries operated at high potentials. On the other hand, from FIG. 6, the charging and discharging curves of the lithium ion secondary battery of Comparative Example I are understood to be shifted toward the low capacity side associated with increase in the number of cycles performed. From this result, the conventional electrolytic solution containing the organic solvent by a mole ratio of about 10 relative to LiPF₆ is considered to be inferior to the electrolytic solution of the present invention, as an electrolytic solution for batteries operated at high potentials.

1. An electrolytic solution containing
a heteroelement-containing organic solvent at a mole ratio of not greater than 1.5 relative to a metal salt,
the heteroelement-containing organic solvent containing a linear carbonate represented by general formula (1) below,
the metal salt being a metal salt whose cation is an alkali metal, an alkaline earth metal, or aluminum and whose anion has a chemical structure represented by general formula (2) below:
R¹⁰OCOOR¹¹ general formula (1)
(R¹⁰ and R¹¹ are each independently selected from CₙHₐF_{b}Cl_{c}Br_{d}Iₑ that is a linear alkyl, or CₘH_{f}F_{g}ClₕBrᵢIⱼ that includes a cyclic alkyl in a chemical structure thereof. “n” is an integer not smaller than 1, “m” is an integer not smaller than 3, and “a”, “b”, “c”, “d”, “e”, “f”, “g”, “h”, “i”, and “j” are each independently an integer not smaller than 0 and satisfy 2n+1=a+b+c+d+e and 2m=f+g+h+i+j.)
(R²¹X²¹)(R²²SO₂)N general formula (2)
(R²¹ is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.
R²² is selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; CN; SCN; or OCN.
R²¹ and R²² optionally bind with each other to form a ring.
X²¹ is selected from SO₂, C═O, C═S, R^{a}P═O, R^{b}P═S, S═O, or Si═O.
R^{a} and R^{b} are each independently selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; OH; SH; CN; SCN; or OCN.
R^{a} and R^{b} each optionally bind with R²¹ or R²² to form a ring.)

2. The electrolytic solution according to claim 1, wherein
the heteroelement-containing organic solvent contains the linear carbonate by not less than 80 vol % or not less than 80 mole %.

3. An electrolytic solution containing
a linear carbonate at a mole ratio of not greater than 1.5 relative to a metal salt,
the linear carbonate being represented by general formula (1) according to claim 1,
the metal salt being a metal salt whose cation is an alkali metal, an alkaline earth metal, or aluminum and whose anion has a chemical structure represented by general formula (2) according to claim 1.

4. The electrolytic solution according to claim 1, wherein
the chemical structure of the anion of the metal salt is represented by general formula (2-1) below.
(R²³X²²)(R²⁴SO₂)N general formula (2-1)
(R²³ and R²⁴ are each independently CₙHₐF_{b}Cl_{c}Br_{d}Iₑ(CN)_{f}(SCN)_{g}(OCN)ₕ.
“n”, “a”, “b”, “c”, “d”, “e”, “f”, “g”, and “h” are each independently an integer not smaller than 0 and satisfy 2n+1=a+b+c+d+e+f+g+h.
R²³ and R²⁴ optionally bind with each other to form a ring, and in that case, satisfy 2n=a+b+c+d+e+f+g+h.
X²² is selected from SO₂, C═O, C═S, R^{c}P═O, R^{d}P═S, S═O, or Si═O.
R^{c} and R^{d} are each independently selected from: hydrogen; a halogen; an alkyl group optionally substituted with a substituent group; a cycloalkyl group optionally substituted with a substituent group; an unsaturated alkyl group optionally substituted with a substituent group; an unsaturated cycloalkyl group optionally substituted with a substituent group; an aromatic group optionally substituted with a substituent group; a heterocyclic group optionally substituted with a substituent group; an alkoxy group optionally substituted with a substituent group; an unsaturated alkoxy group optionally substituted with a substituent group; a thioalkoxy group optionally substituted with a substituent group; an unsaturated thioalkoxy group optionally substituted with a substituent group; OH; SH; CN; SCN; or OCN.
R^{c} and R^{d} each optionally bind with R²³ or R²⁴ to form a ring.)

5. The electrolytic solution according to claim 1, wherein
the chemical structure of the anion of the metal salt is represented by general formula (2-2) below.
(R²⁵SO₂)(R²⁶SO₂)N general formula (2-2)
(R²⁵ and R²⁶ are each independently CₙHₐF_{b}Cl_{c}Br_{d}Iₑ.
“n”, “a”, “b”, “c”, “d”, and “e” are each independently an integer not smaller than 0 and satisfy 2n+1=a+b+c+d+e.
R²⁵ and R²⁶ optionally bind with each other to form a ring, and in that case, satisfy 2n=a+b+c+d+e.)

6. The electrolytic solution according to claim 1, wherein
the metal salt is (CF₃SO₂)₂NLi, (FSO₂)₂NLi, (C₂F₅SO₂)₂NLi, FSO₂(CF₃SO₂)NLi, (SO₂CF₂CF₂SO₂)NLi, or (SO₂CF₂CF₂CF₂SO₂)NLi.

7. The electrolytic solution according to claim 1, wherein
the linear carbonate is represented by general formula (1-1) below.
R¹³OCOOR¹⁴ general formula (1-1)
(R¹³ and R¹⁴ are each independently selected from CₙHₐF_{b} that is a linear alkyl or CₘH_{f}F_{g} that includes a cyclic alkyl in a chemical structure thereof. “n” is an integer not smaller than 1, “m” is an integer not smaller than 3, and “a”, “b”, “f”, and “g” are each independently an integer not smaller than 0 and satisfy 2n+1=a+b and 2m=f+g.)

8. The electrolytic solution according to claim 1, wherein
the linear carbonate is selected from dimethyl carbonate, ethyl methyl carbonate, or diethyl carbonate.

9. The electrolytic solution according to claim 1, wherein
the mole ratio is in a range of 1 to 1.5.

10. A secondary battery provided with the electrolytic solution according to claim 1.

11. The secondary battery according to claim 10, provided with a positive electrode active material whose reaction potential is not lower than 4.5 V versus Li reference.

12. The secondary battery according to claim 10, provided with a lithium metal complex oxide having a spinel structure as the positive electrode active material.

13. The secondary battery claim 10, provided with a positive electrode current collector made from aluminum.
